(19) United States
(12) Patent Application Publication
(10) Pub. No.: US 2025/0072441 A1
(43) Pub. Date: Mar. 6, 2025

(54) METHOD FOR PREPARING A COMESTIBLE NUTRIENT COMPOSITION AND USE THEREOF

(71) Applicant: The Cultivated B. GmbH, Heidelberg (DE)

(72) Inventors: Yuichiro Ueda, Heidelberg (DE); Jitin Bali, Heidelberg (DE); Gerrit Daubner, Heidelberg (DE); Matheus Kolodziej, Heidelberg (DE); Ilya Galperin, Heidelberg (DE); Natália Bittencourt Melani, Heidelberg (DE); Nicolai Bluthardt, Heidelberg (DE)

(73) Assignee: The Cultivated B. GmbH, Heidelberg (DE)

(21) Appl. No.: 18/724,743

(22) PCT Filed: Jan. 30, 2023
(86) PCT No.: PCT/EP2023/052154

§ 371 Date: Jun. 27, 2024

(30) Foreign Application Priority Data
Jan. 31, 2022 (EP) 22154336.6

(51) Int. Cl. A23L 2/42; A23L 2/38; A23L 2/39; C12N 5/071

(52) U.S. Cl. CPC A23B 70/00; A23L 2/382; A23L 2/39; C12N 5/0602; C12N 2500/92

(57) ABSTRACT

The invention relates to a method for preparing a comestible nutrient composition comprising the steps of (i) cultivating proliferating non-human animal cells of interest in vitro until the cell count of said cells has multiplied by at least 2-fold or more; (ii) harvesting the cells to provide cell-derived material; and (iii) preserving the cell-derived material from the cells harvested in step (ii) from spoiling. Furthermore, the invention refers to a comestible nutrient composition obtainable from such method and use thereof for providing a well-defined nutrient composition.

The present invention relates to a method for preparing a comestible nutrient composition comprising the steps of (i) cultivating proliferating non-human animal cells of interest in vitro until the cell count of said cells has multiplied by at least 2-fold or more; (ii) harvesting the cells to provide cell-derived material; and (iii) preserving the cell-derived material from the cells harvested in step (ii) from spoiling. Furthermore, the invention refers to a comestible nutrient composition obtainable from such method and use thereof for providing a well-defined nutrient composition.

There is an increasing global demand for comestible products that provide nutrient values such as high protein content and healthy nutrient contents. This demand is often met by products of slaughtered animals such as meat. Meat often contains comparably high contents of valuable nutrient such as vitamins (e.g., vitamin B1 (thiamin), vitamin B3 (niacin), vitamin B6 and vitamin B12), meat specific amino acids such as lysine, methionine, isoleucine or tryptophan which are not existing in plant based comestible nutrient compositions, selenium, and pantothenic acid. Furthermore, liver tissue often additionally comprises high contents of nutrients vitamin A, copper. collagen, polylysine, hyaluronic acid.

However, there are increasing ethical and economic constraints and concerns when using meat from slaughtered animals. Undesirable material such as xenobiotics, hormones and precursors thereof and high caloric ingredients are inevitably present in animal-derived meat. The nutrient contents, including macronutrient (e.g., proteins, fatty acids, and carbohydrates, and dietary fiber) and micronutrients composition (e.g., minerals, vitamins), are not controllable and adjustable as desired. In addition, high greenhouse gas production and environmental pollution (e.g., eutrophication) and high area consumption are often associated with meat production. For an increasing population of >7 billion human being in the world, it will hardly be possible or even desirable to provide all with meat.

Therefore, there is a considerable interest for products that replace meat from slaughtered animals.

In 2013, a hamburger based on cell-culture material from cultivated bovine myocytes has been introduced. Even solid meat replacements that imitate whole parts of animal muscle tissue are considered. In this context, it is considered to produce pig meat either from skeletal muscle cells or pig embryonic cells cultivated as stem cells, matured into myofibers and combined with scaffolds to obtain a meat-like product (cf. Kurt et al., Austin Food Sci, 2021, 6(1):1041). A number of advantages has been found to obtain cell-culture-derived meat from chicken cells, duck cells, beef cells and pork cells (cf. Hong et al., Food Sci. of Anim. Resour., 2021, 41(3):355-372), such as that clean and safe meat was provided. It took, however, high efforts to prepare such material and the costs were very high. 3D shaping is energy and resource demanding and typically required scaffold structures.

Furthermore, a solid meat-like product is not suitable for individuals who have problems to chew and/or swallow such as elderly persons and patients suffering from dysphagia. For these individuals common protein shakes, which are often derived from certain purified proteins or milk are known, such as high-protein milk protein concentrates (cf. Sikand et al., J. Diary Sci., 2011, 94:6194-6202).

In case that a patient cannot eat any or enough food because of e.g. a pathologic situation associated with difficulties in chewing and/or swallowing such as dysphagia, because of a surgery that interferes with eating, or because of decreased appetite, enteral nutrition (e.g., nasogastric nutrition (i.e., via a tubing through the nose into the stomach or small intestine also designated as nasogastric intubation), gastrostomy (i.e., via a tubing through the skin into the stomach), jejunostomy (i.e., via a tubing through the skin into the jejunum), or other type of tube feeding with such protein composition may be considered. In such case, the administered composition may contain protein, carbohydrates (sugar), fats, vitamins and minerals, given through a tube into the stomach or small intestine. Exemplified compositions are described in Malone, 2005 (“Enteral Formula Selection: A Review of Selected Product Categories”, in Nutrition Issues in Gastroenterology, Series #28, ed. Carol Rees Parrish, Practical Gastroenterology).

Protein compositions usable for the aforementioned purposes have the disadvantage that the nutrient content is not meat-like and valuable nutrients such as minerals, essential amino acids (e.g., lysine, isoleucine, tryptophan, etc.) and vitamins that are typical for meat are missing. Rather, such protein compositions known for the above-referenced purposes typically are derived from milk (e.g., containing milk, skimmed milk, milk protein concentrate, casein, caseinates, lactalbumin) or derived from plant material (e.g., containing soy protein isolate, whey protein concentrate) or hydrolysates or isolates thereof as described in Malone, 2005. This may be sub-optimal for human an animal nutrition.

Furthermore, the compositions known in the art have the drawback that many subjects have intolerance towards soy, wheat or milk products that represent the key sources of proteins in protein compositions known in the art. This is of particular interest in the context of elderly people and enteral nutrition.

There is still an unmet need for comestible products that can replace meat, can provide a beneficial and adjustable nutrient content and can, concomitantly be efficiently prepared.

Surprisingly, it has been found that a comestible nutrient composition can be efficiently prepared on the basis of a cell culture that is preserved against spoiling in a subsequent step and that may be optionally mixing the cell-derived material of step (iii) with one or more further consumable ingredients.

An aspect of the present invention relates to a method for preparing a comestible nutrient composition comprising the steps of:
(i) cultivating proliferating non-human animal cells of interest in vitro until the cell count of said cells has multiplied by at least 2-fold or more;
(ii) harvesting the cells to provide cell-derived material; and
(iii) preserving the cell-derived material from the cells harvested in step (ii) from spoiling; and
(iv) optionally mixing the cell-derived material of step (iii) with one or more further consumable ingredients selected from the group consisting of one or more vitamins, one or more minerals, one or more aroma compounds, one more food colors, one or more types of fibers, ethanol, acetic acid, carbonic acid, and combinations of two or more thereof.

The comestible nutrient composition obtainable (or obtained) from such method may have potential benefits such as, e.g., supporting weight loss, boosted metabolic rates, detoxification of the body, and improved organ functions. The nutrient contents, including macronutrient (e.g., proteins, fatty acids, and carbohydrates, and dietary fiber) and micronutrients composition (e.g., minerals, vitamins, amino acids), may be well controllable and adjustable as desired. Undesirable material such as xenobiotics (e.g. residual drugs such as antibiotics), hormones and precursors thereof, pathogens, and high caloric ingredients may be widely avoided by using controlled laboratory environment conditions. Thus, particularly clean conditions in preparations and of the comestible nutrient composition may be achieved. The usability of chemically and biochemically defined cell culture medium and cells allows a stable and reliable production widely independent on external factor. The aforementioned benefits are superior in comparison to natural animal-based products such as meat. Further, a comestible nutrient composition of the present invention is prepared in a particularly efficient manner.

The comestible nutrient composition obtainable (or obtained) from such method may also be suitable for nutrition of elderly people as well as for enteral nutrition (also: enteral feeding, such as, e.g., nasogastric nutrition such as nasogastric intubation, gastrostomy, jejunostomy, or other type of tube feeding).

The comestible nutrient composition may contain cell-derived material from (non-human) animal cells for designing drinks that would cater to various demographics and dietary preferences. For this process, only a small number of cells are needed as source material. This may save resources dramatically and is considerable as more sustainable. Less landscape/space is required to prepare 1 kg of cell biomass of a comestible nutrient composition of the present invention than for 1 kg of cell biomass of animal meat. Less water is consumed. In addition, water recycling is and efficient waste management is possible.

Released undesired emissions such as methane gas emissions are typically significantly lower in comparison to those released in production of comparable amounts of animal-based cell production such as meat.

The method of the present invention may be scaled up to a desired degree.

It is enabled to fulfill individual demands and digestion of the comestible nutrient composition is more effectively (easier) than that of a comparable piece of meat. The nutrient content may be freely adaptable. To an individual's nutrient demands. The comestible nutrient composition may allow a highly efficient or even optimal path to absorb valuable nutritional components superior to other administration routes.

Accessibility and resorption may be improved. This may have beneficial impact in medical sector. In this context, also prebiotics and even drug agents (including ribonucleic acid-based drugs such as silencer RNA (siRNA)) may be added and, thus, applied to a consumer.

As used in the context of the present invention, the term “comestible” may be understood as generally understood in the art as consumable without (substantial) health risk when consuming reasonable amounts as indicated herein. Preferably, “comestible” means components suitable for animal and, in particular human, food consumption. This may also be interpreted in the context of accreditation of official regulatory offices.

(Non-human) animal cells and their extracellular matrices (ECMs) components may be prepared, optionally along with further consumable ingredients (also “additives”) as a cell-based drink from clean and controlled bioprocessing which would suit the individual lifestyle and even clinical and therapeutic demands as a new form of nutritional consumable/supplement.

As used herein, the terms “drink” and “beverage” may be understood interchangeably in the broadest sense as generally understood in the art as a liquid or syrup-like orally consumable composition. It may or may not contain alcohol. Optionally, the comestible nutrient composition may comprise an ethanol/water mixture. In certain embodiments of the present invention, it may comprise 0 to 80% by weight, 0 to 40% by weight, 0 to 6% by weight, 1 to 15% by weight, 5 to 15% by weight or 10 to 40% by weight, based on the total composition, of ethanol. Optionally, the comestible nutrient composition may comprise a water/fat emulsion. In a preferred embodiment, the comestible nutrient composition may comprise at least 0.5% by weight, at least 1% by weight, at least 2% by weight, at least 5% by weight, at least 10% by weight, or at least 25% by weight, based on the total weight of the composition, of cell-derived material.

The method of the present invention may also be considered as a bioprocessing or cellular agricultural method. It may be considered as being based on cultivated cells as consumables. It may provide cell-based nutrition.

The taste of the comestible nutrient composition may be sweet, salty, bitter, sour, umami, or a combination of two or more thereof.

### Step (i) of Cultivating Proliferating Non-Human Animal Cells of Interest

As used herein, the term “cells of interest” may be understood as the one or more cell types that are intended to be used in the process. Preferably, material derived from these cells should be contained in a comestible nutrient composition. The cells of interest may be any non-human animal cells.

In a preferred embodiment, the cells of interest are:
myocytes or precursor cells thereof, in particular (myo-)satellite cells;
adipocytes or precursor cells thereof;
hepatocytes or precursor cells thereof;
induced pluripotent stem cells (iPSCs);
non-human embryonic stem cells;
or a combination of two or more thereof.

The cells of interest may be obtained by any means. When using primary cells, a piece of tissue may be obtained from a donor animal or animal-derived material (e.g., egg, placenta (reproductive tissue), skin, and/or hair). Optionally, this is washed and/or cut into smaller pieces. Optionally, the cells may be at least partly separated from one another by shear forces and/or enzymes (e.g., collagenase I and/or dispase and/or (chymo)trypsin) and/or a hypertonic solution (e.g., 1 mM sodium citrate in potassium/sodium chloride of 570 mOsm/kg final osmolality. Optionally, the cells may be further treated with a complexing agent (e.g., ethylenediaminetetraacetic acid (EDTA) and/or sodium citrate). Optionally, the cells may be suspended in a suspension medium (e.g., phosphate buffered saline (PBS)). Optionally, one or more antibiotics (e.g., penicillin, streptomycin, etc.) may be added. Optionally, the cells may be subjected to centrifugation/resuspension cycles for washing. The cells may be added to a suitable culture conditions.

Adult stem cells such as, e.g., satellite cells may be obtained by any means. For instance such adult stem cells may be obtained as described in Schmidt et al. (Cellular and Molecular Life Sciences, 2019, 76:2559-2570), Hindi et al. (Science Signaling, 2013, 6(272):DOI: 10.1126/scisignal.2003832), Choi et al. (Food Sci. Anim. Resour., 2020, 40(5):852-859), and/or Nie et al. (PlosONE, 2014, 9(1):e88012). This may optionally include cell factors such as, e.g., Pax3 and/or Pax7 for satellite cells, and/or MyoD and/or Myf5 for myoblasts. Myocytes may optionally be detected by myogenin. Myotubes may be positive for Myosin Heavy Chain (MyHC).

Furthermore, mature myotubes may be multinucleated which may exemplarily be detected using nuclear-specific dyes like DAPI and Hoechst. Optionally, magnetic-activated cell sorting (MACS) may be used to separate cells from one another and/or to isolate a certain desired cell type (e.g., CD29-positive satellite cells by using CD29 as cell marker, identifiable by immunostaining with CD29 and/or Pax7). Cells (e.g., satellite cells) may optionally be subjected to an ice-cold treatment as described in Benedetti et al. (SkeletalMuscle, 2021, 11(7):doi.org/10.1186/s13395-021-00261-w), thus, an ice-cold buffer (e.g., ice-cold PBS) may be optionally added to a flask when passaging the cells.

In a preferred embodiment, the cells of interest are or comprise myocytes or precursor cells thereof, in particular (myo-)satellite cells. For example, bovine satellite cells may be cultivated under conditions as described in the art (cf., Skrivergaard et al., Int. J. Mol. Sci., 2021, 22:8376). In one embodiment cells such as myocytes or precursor cells thereof (e.g., (myo-)satellite cells) or other cell types may be harvested from skeletal muscle tissue. This is exemplified in the experimental section below.

In a further embodiment, cells may originate from eggs (e.g., avian eggs such as, e.g., chicken eggs). Egg cells may be harvested before hatch (e.g. a ca. 21 days old or younger chicken embryo. This allows obtaining cells at any developmental stage such as embryonic stem cell, embryonic germ stem cell, progenitor cell, and fully developed/functional cell.

In a further embodiment, cells may originate from bone marrow. From bone marrow, high quantities of mesenchymal stem cells can be harvested. Mesenchymal stem cell may, for instance, be differentiated into myocyte, adipocyte, osteoblast, neuron, and chondrocyte cells. Mesenchymal stem cells may secrete one or more beneficial growth factors/cytokines for culturing cells as well. In a further embodiment, cells may originate from feather from poultry, e.g., as follicle cells, which may have similar characteristics like mesenchymal stem cell.

In a further embodiment, cells may be harvested from any organs/tissues of a living being of any biological kingdom such as, e.g., an organ or tissue of one or more animals, one or more plants, one or more fungi, one or more protista, one or more eubacteria, and/or one or more archaebacteria for cultivation to proliferate cells as well as obtain macro- and micro-nutrients. Such cells may be added to the comestible nutrient composition of the present invention.

The cells of interest may be primary cells or cells of a permanent cell culture. In one embodiment, the cells of interest are primary cells. In another embodiment, the cells of interest are permanent cell culture.

Primary cells may originate from a biopsy or necropsy. A cell culture may be established. A permanent cell culture may also be (an optionally commercial) cell bank. A permanent cell culture may comprise immortalized cell lines. Optionally, induced pluripotent stem cells (iPSCs) and/or non-human embryonic stem cells may be induced to form the desired cell type. Such step may also be included in the present invention. In one embodiment, the cells of interest are somatic mature cells. Slaughtering animals may be reduced or avoided. This may be desirable for ethical reasons and animal welfare.

The cells of interest may originate from any non-human animal species (“donor”). For example cells of interest may originate from a mammal (mammalian animal) (e.g., sheep, cow/beef, goat, pig, buffalo, deer, horse, camel, rabbit, etc.), from a bird species (poultry, avian cells)(e.g., duck, chicken, goose, turkey, goose, pigeon, fowl, etc.), from a reptile (e.g., alligator, crocodile, snake etc.), fish (e.g., salmon), or from an insect (e.g., mealworm, bee larva, etc.). Avian cells are used for vaccine production, this may allow obtaining scalable in-suspension serum-free produced cells from commercial sources at comparably large scales.

In one embodiment, cells of interest may originate from a ruminant such as, e.g., may be selected from the group consisting of cow, sheep, goat, deer, and buffalo cells. In one embodiment, cells of interest may originate from a non-ruminant such as, e.g., may be pig cells. In one embodiment, cells of interest may originate from a pseudo-ruminant such as, e.g., may be selected from the group consisting of horse, camel, and rabbit cells. The cells of interest may originate from any non-human animal species (“donor”).

In one embodiment, cells of interest may be primary cells originating from a mammal (mammalian animal), from a bird species, from a reptile, a fish, or from an insect. In another embodiment, cells of interest may be cells from a permanent mammal, bird, reptile, fish, or insect cell culture. In one embodiment, the cells used in the context of the present invention are primary mammalian cells (also: mammal cells). In one embodiment, the cells used in the context of the present invention are from a mammalian cell culture (also: mammal cell culture).

Cells may optionally be stored as a cell stock at any condition usable for such purpose such as in a refrigerator (e.g. at 1 to 10° C., e.g., (approximately) 4° C.), in a freezer (e.g. at −30 to 0° C., e.g., (approximately) −20° C.), in a high-performance freezer (e.g. at −90 to −70° C., e.g., (approximately) −80° C.), or in liquid nitrogen (N₂). Optionally, a frozen stock may or may not contain dimethyl sulfoxide (DMSO).

Optionally, cells of different cell types and/or different species may be combined with each other. This may optimize the nutrient contents of the comestible nutrient composition.

For example, cells may be combined with each other, wherein cells of at least two different cells types may be combined with each other. These may be optionally from the same or different species origin. For example, the following cells may be combined with each other, wherein cells of at least two different species are combined with each other, for example:
cells from two or more different species of birds,
cells from two or more different species of mammals,
cells from two or more different species of fish,
cells from two or more different species of reptile,
cells from two or more different species of insects,
cells from at least one bird species and from at least one mammal species,
cells from at least one bird species and from at least one fish species,
cells from at least one bird species and from at least one reptile species,
cells from at least one bird species and from at least one insects species,
cells from at least one mammal species and from at least one fish species,
cells from at least one mammal species and from at least one reptile species,
cells from at least one mammal species and from at least one insects species,
cells from at least one fish species and from at least one reptile species, or
cells from at least one fish species and from at least one insects species.

The cell count ratio of two cell types may be at any range. For example, it may be in the range of from 1:1 to 1:1000, of from 1:1.5 to 1:500, of from 1:2 to 1:200, of from 1:3 to 1:100, of from 1:5 to 1:50, or of from 1:5 to 1:10.

For example, cells may be combined with each other, wherein cells of at least three different cells types may be combined with each other. These may be optionally from the same or different species origin. For example, cells may be combined with each other, wherein cells of at least three different species are combined with each other, for example:
cells from three or more different species of birds,
cells from three or more different species of mammals,
cells from three or more different species of fish,
cells from three or more different species of reptile,
cells from three or more different species of insects,
cells from at least two bird species and from at least one mammal species,
cells from at least two bird species and from at least one fish species,
cells from at least two bird species and from at least one reptile species,
cells from at least two bird species and from at least one insect species,
cells from at least two mammal species and from at least one bird species,
cells from at least two mammal species and from at least one fish species,
cells from at least two mammal species and from at least one reptile species,
cells from at least two mammal species and from at least one insect species,
cells from at least two fish species and from at least one bird species,
cells from at least two fish species and from at least one mammal species,
cells from at least two fish species and from at least one reptile species,
cells from at least two fish species and from at least one insect species,
cells from at least two reptile species and from at least one bird species,
cells from at least two reptile species and from at least one mammal species,
cells from at least two reptile species and from at least one fish species,
cells from at least two reptile species and from at least one insect species,
cells from at least two insect species and from at least one bird species,
cells from at least two insect species and from at least one mammal species,
cells from at least two insect species and from at least one fish species, or
cells from at least two insect species and from at least one reptile species.

The cell count ratio of three cell types may be at any range. For example, it may be in the range of from 1:1:1, 2:1:1, 2:2:1, 3:2:1, 4:2:1, 4:2:1, 4:3:1, 4:4:1, 4:2:1, 4:1:1, 4:3:2, 4:4:2, 5:1:1, etc.

Such cells of different species origin may be of the same or different cell origin. In one embodiment, all cells are of the same cell type origin. In another embodiment, the cells are of different same cell type origin. In one embodiment, all cells are muscle cells (in particular (myo-)satellite cells). For instance, muscle cells (in particular (myo-)satellite cells) from poultry and mammals may be admixed. For instance, muscle cells (in particular (myo-)satellite cells) from poultry and fish may be admixed. For instance, muscle cells (in particular (myo-)satellite cells) from pork and salmon may be admixed. For instance, muscle cells (in particular (myo-)satellite cells) from beef and salmon may be admixed. For instance, muscle cells (in particular (myo-)satellite cells) from pork and salmon may be admixed. For instance, muscle cells (in particular (myo-)satellite cells) from pork and beef cells may be admixed. For instance, muscle cells (in particular (myo-)satellite cells) from pork, salmon and beef may be admixed (e.g., in a 1:1:1 ratio).

The cells may be cultivated at any conditions suitable for cell proliferation. In a preferred embodiment, cultivating the cells is conducted until the cell count of said cells has multiplied by at least 2-fold or more, 3-fold or more, 4-fold or more, 5-fold or more, 10-fold or more, or 25-fold or more.

Preferably, the cells of interest are cultured under sterile and controlled conditions to prevent contamination and provide beneficial growth conditions. External factors such as temperature, pH and humidity may be controlled. Cultivation may be performed at laboratory-grade incubators or in industrial large scale. For mammalian cells, cells growth conditions may, for example be: 37° C., approximately 5% by weight CO₂, approximately 95% air, and approximately 85-95% relative humidity. The culturing conditions may be adapted to the respective cells of interest. For avian cells, culturing temperature may be 40 to 42° C. For example, at laboratory scale, cells may be cultivated in glass or plastic petri dishes, glass flasks, or plastic wears such as plastic flasks. Typically, a pH range is cellular physiological range (pH 7.0-7.4). Optionally, a cell culture device may be pre-coated (e.g with a protein such as, e.g., collagen (type 1), gelatin), which may improve initial attachment of the cells.

Also, bioreactors may be used for cultivating the cells. This may be any type of bioreactor. For instance, a suitable bioreactor may be a stirred tank bioreactor, a wave bioreactor, an airlift bioreactor, a packed bed bioreactor, a vertical wheel bioreactor, or a hollow fiber bioreactor.

The method of the present invention may be scaled up to a desired degree. A bio reactor may have any size such as a volume of below 10 L, of from 10 to 50 L, of from 20 to 100 L, of from 50 to 250 L, of from 100 to 1000 L, or above 1000 L.

The cultivating conditions may be controlled (e.g., pH, gas, temperature, etc.). An advantage of using bioreactors over traditional 2D monolayer cultures may be is its ability to increase cell density, with optionally up to 10⁹ cells/mL of cell culture medium.

The cells may be cultivated as suspended cells, as adherent cells or as semi-adherent cells or a combination thereof. Optionally, in the case of adherent cells, the surface may be treated by plasma treatment, collagen coating, etc., to improve initial attachment of cell to either reach proliferate state (dividing and increase in size) or terminally differentiated state or rest-phase state to achieve cellular monolayer for subsequent growth steps (attached on the surface).

Any suitable cell culture medium suitable for the respective cell type may be used. Typically, the cell culture medium suitable for proliferating animal cells comprises one or more carbon sources such as sugars. Optionally, the cell culture medium may comprise supplements such as, e.g., amino acids, fatty acids, vitamins, minerals, growth factors, trace elements, etc. The person skilled in the art will be aware of suitable cell culture media for a number of cell types. For instance for (myo-)satellite cells, B8 and Beefy-9 medium with minimized components such as DMEM/F-12 medium as a basal medium combined with recombinant insulin, L-ascorbic acid 2-phosphate, recombinant transferrin, sodium selenite, NRG1, FGF2, TGF-β1, HEPES, and bicarbonate may be used and may be modified accordingly to the specific growth conditions.

Optionally, medium components such as, e.g., glucose, amino acid, metabolites: lactate, ammonia, active growth factors: FGF2, and/or TFG-b1, may be measured and controlled during cultivating.

The cell culture medium may or may not comprise fetal bovine serum (FBS) (also: fetal calf serum (FCS)). The cell culture medium may or may not comprise animal-derived serum. In a preferred embodiment, the step (i) is cultivating performed in the absence of animal-derived serum. Fetal bovine serum (FBS) is commonly used for traditional cell culture with 5-20% by weight to reduce the total cost of cultivating cells. Drawbacks of using FBS are multiple and include slaughtering of fetal bovine, possible contamination originating from an animal source such as germs and pathogens, and high processing and storage cost.

As indicated above, a beneficial technical effect of the present invention may be the ability to control the ingredient content in the comestible nutrient composition. Thus, in a preferred embodiment, the method comprises the step of adjusting the content ratios and types of the cells cultivated in step (i) to obtain a defined nutrient content of interest, in particular a defined protein content of interest. Thus, depending on the type of cells used as cells of interest, the type and content ranges of proteins of interest and optionally their metabolic products (e.g., peptides and amino acids) may be adjusted.

### Step (ii) of Harvesting the Cells

The step (ii) of harvesting the cells to provide cell-derived material may be conducted by any means known in the art. When the cells are cultivated as adherent cells or as semi-adherent cells, the step (ii) of harvesting the cells to provide cell-derived material may comprise detaching the cells from the substrate. This may be performed mechanically (e.g., by scratching) or supported by one or more enzymes (e.g, trypsin) as generally known in the art. A cell suspension may be obtained.

In one embodiment, the step (ii) comprises administering a solution containing a citrate salt (e.g., sodium citrate) to the cells to be harvested. In one embodiment, the solution containing a citrate salt (e.g., sodium citrate) is a buffer solution, in particular phosphate buffered saline (PBS). In one embodiment, the citrate salt is a water soluble citrate salt. In one embodiment, the citrate salt is sodium citrate. In one embodiment, the solution containing a citrate salt (e.g., sodium citrate) is administered to the cells for 2-20, 5-10, or 10-15 minutes before flushing the cells. Thereby, the cells may be detached from their substrate, in particular when the cells have been grown (at least partly or completely) adherently in step (i). The concentration of citrate salt (e.g., sodium citrate) may be adjusted. In one embodiment, the concentration of citrate salt (e.g., sodium citrate) may be in the range of 0.01 to 10 mM, or 0.05 to 5 mM, or 0.1 to 4 mM, or 0.2 to 3 mM, or 0.5 to 5 mM, or (approximately 1 mM, of citrate ions (including fully deprotonated citrate, hydrogencitrate and dihydrogencitrate). In one embodiment, sodium citrate may be used in the range of 0.01 to 10 mM, or 0.05 to 5 mM, or 0.1 to 4 mM, or 0.2 to 3 mM, or 0.5 to 5 mM, or (approximately 1 mM.

In one embodiment, the step (ii) of harvesting the cells further comprises at least partly separating the cells from the cell culture medium. This may be achieved by any means. In a preferred embodiment, the step (ii) includes the removal of cell culture medium. In such step, at least 10% by weight, at least 25% by weight, at least 50% by weight, at least 70% by weight, at least 80% by weight, at least 90% by weight, at least 95% by weight, at least 98% by weight, or at least 99% by weight, referred to the total amount of cell culture medium present at the end of cultivating step (i), or (essentially) all cell culture medium present at the end of cultivating step (i), may be removed.

The at least partly removal of the cell culture medium from the cells may be achieved by any means. In a preferred embodiment, the step (ii) includes the removal of cell culture medium by means of centrifugation, filtration, dialysis, or rinsing adherent cells. Such step may optionally include optional washing steps with one or more buffers. Such washing step may also remove remaining and metabolites. In a preferred embodiment, the step (ii) includes the removal of cell culture medium by means of centrifugation and optional washing steps with one or more buffers.

Optionally, the removed cell culture medium or water thereof may be recycled. Optionally, it may be used in the process as cell culture medium in step (i). A waste management may be used to further use the nutrients contained in the consumed cell culture medium. Thus, optionally, also the not yet consumed nutrients may recycled to step (i).

In an alternative embodiment, the step (ii) includes the maintenance of at least parts of cell culture medium and a step of pasteurization.

Optionally, the cells may be resolubilized such as in a buffered solution. Preferably the one or more buffer agents contained in such buffer are consumable at moderate concentrations. Optionally, such buffered solution may contain a buffer agent selected from the group consisting of histidine, acetate, citrate, glycine, phosphate, or tris(hydroxymethyl)aminomethane (TRIS).

Optionally, the step (ii) of harvesting the cells to provide cell-derived material comprises a further step of rendering at least a part of the cells inviable, in particular breakup the cells. This may be achieved by any means, such as, e.g., freeze-thaw cycles, lyophilization, applying ultrasound, mechanical stress (e.g, in a French press or a homogenizer. This may provide a cell-derived material that contains dead cell-derived material. Such dead cell-derived material may comprise cell membranes and fractions thereof and inner parts of the cells such as cell lysate, including cytoplasm and/or cellular organelles.

In a preferred embodiment, the released proteins are preferably at least partly dissolved in the comestible nutrient composition. As proteins may be susceptible to or even unstable in precipitation close to the isoelectric point, such pH ranges are preferably avoided. In a preferred embodiment, the method of the present invention does not comprise discarding certain parts of the cells. In a preferred embodiment, the cell-derived material and the finally obtained comestible nutrient composition) comprises cell membranes and fractions thereof and inner parts of the cells such as cell Iysate, including cytoplasm and/or cellular organelles. In a preferred embodiment, the cell-derived material (and the finally obtained comestible nutrient composition) comprises (essentially) the full content of the cells.

In a preferred embodiment, the cells of interest may be myocytes. Proteins of interest may be those present in the comestible nutrient composition which may be myofibrillar proteins. Myofibrillar proteins are the most common proteins in muscle tissue and have an isoelectric point close to pH 5.5. Accordingly, the pH range of approximately 5.5 (e.g., pH 5-6, preferably pH 5.2-5.8, in particular pH 5.4-5.6) is preferably avoided.

Optionally, the cell-derived material may or may not be treated with ultrasound (e.g., as described in Chen et al., Ultrasonics Sonochemistry, 2021, 76:105652).

A buffer for maintaining proteins in solution may optionally comprise additives that stabilize the solution. For example, such buffer may comprise a salt (e.g., sodium chloride) to stabilize proteins (e.g., myofibrillar proteins). Optionally, the salt (e.g., sodium chloride)/dry cell-derived material mass ratio may be set to 100:4-4.5 to ensure protein solubility in the liquid. Alternatively or additionally, stabilizing agents such as tripolyphosphate, tetrasodium pyrophosphate, tetrapotassium pyrophosphate, sodium tripolyphosphate, or potassium tripolyphosphate, or combinations of two or more thereof may be added to increase the solubility of proteins (e.g., myofibrillar proteins).

In an alternative embodiment, viable cells may be harvested. This may optionally be achieved by cultivating cells in a suspension culture. Then, the cells including the cell culture medium may be further used as cell-derived material.

In an alternative embodiment, viable cells may be harvested by applying moderate centrifugal forces or filtration at low shear stress to cells either cultivated in a suspension culture or attached cells previously detached by mild means such as enzymes. Cells may also be harvested as described in Nienow et al., (Biochemical Engineering Journal, 2014, 85:79-88).

In one embodiment, the cells or fragments thereof are separated from the medium and represent the cell-derived material without the cell-culture medium. In another embodiment, the cell-derived material still contains at least parts of the cell culture medium.

Step (iii) of Preserving the Cell-Derived Material from Spoiling

The step (iii) of preserving the cell-derived material from spoiling may be conducted by any means. For example, the cell-derived material may be lyophilized, fermented, pickled, smoked, or dried to avoid microbial growth. Alternatively or additionally, the product may be heated, while reducing sugars such as glucose, lactose, and maltose may optionally be added to achieve a Maillard reaction during heating. Alternatively or additionally, preserving agents (e.g., benzoic acid) may optionally be added.

In a preferred embodiment, the step (iii) includes processing the cell-derived material into a powder. This may be achieved by any means known in the art. In a preferred embodiment, processing the cell-derived material into a powder is obtained by at least partly removal of liquid content. This may be achieved by any means known in the art. Processing the cell-derived material into a powder may be achieved by lyophilization or another drying step. In a preferred embodiment, processing the cell-derived material into a powder is obtained by lyophilization.

Lyophilization may be understood in the broadest sense and may include (classical) freeze-drying or spray freeze-drying. Lyophilization may be conducted with or without prior homogenization of the suspension. The procedure of freeze-drying is generally known by the person skilled in the art since decades (cf., U.S. Pat. No. 3,964,174). Freeze-drying is also suitable for preserving meat-derived products such as ham, while essentially maintaining its nutrient content (cf. Ma et al., Saudi Journal of Biological Sciences, 2018, 25:724-732). For example, conditions as described in Ma et al. or U.S. Pat. No. 3,964,174 may be used in the context of the present invention.

Cryoprotectants and lyoprotectants may optionally be added when the lyophilization step is conducted. Cryoprotectants and lyoprotectants may, for instance, include agents selected from the group consisting of trehalose, sucrose, sorbitol, dextran, sodium chloride, disodium sulfate, diammonium sulfate, glycerol, polyphenol, proline, glycine, glutamic acid, histidine, arginine, 4-hydroxyproline, L-serine, β-alanine, lysine, hydrochloride, sarcosine or γ-aminobutyric acid, and combinations of two or more thereof. Furthermore, one or more bulking agents may optionally be added in such lyophilization step. These may improve the mechanical properties and appearance of the final lyophilized product. Bulking agents may optionally be selected from the group consisting of mannitol, lactose, and combinations thereof.

In a preferred embodiment, the cell culture medium is at least partly removed from the cell-derived material harvested in step (ii). In an alternative preferred embodiment, the cell-derived material from the cells harvested in step (ii) still contains cell culture medium and is a liquid or pasty composition.

In a preferred embodiment, the step (iii) includes pasteurization. Pasteurization may be understood in the broadest sense as a method in which the comestible nutrient composition is treated with mild heat, usually to less than 150° C. (e.g., 45 to 150° C., 50 to 125° C., 55 to 100° C., or 60 to 95° C.) to prevent microbial growth. It may be intended to destroy or deactivate organisms and/or enzymes that contribute to spoilage or risk of disease. In a preferred embodiment, the step (iii) includes pasteurization, wherein the cell-derived material from the cells harvested in step (ii) still contains cell culture medium and is a liquid or pasty composition.

In a preserving step (iii) such as, e.g., including lyophilization or drying, may optionally also include a step of adding one or more antioxidants such as, e.g., vitamin D, vitamin E, protein hydrolysates, or sodium sulfite, or combinations of two or more thereof, which may be added to prevent oxidation.

Optionally, a lyophilized or dried product (which may be, e.g., a powder or foam-like) may be milled to form a homogeneous powder. Such optional milling process may be applied using a pulverizer.

Optionally, the obtained dried powder may be the final product (comestible nutrient composition). Alternatively or additionally, such powder may be re-dissolved or resuspended in a suitable liquid such as, e.g, water, an consumable aqueous buffer, or a mixture of one thereof comprising ethanol. Accordingly, on a preferred embodiment, the comestible nutrient composition is a drinkable composition, step (iii) includes processing the cell-derived material into a powder, and the method includes the further step of suspending the powder in an aqueous liquid, in particular in mineral or tap water.

Alternatively or additionally, such powder may be further processed into a granulate.

The product obtained from step (iii) (e.g., a powder or a liquid or a gel) may optionally be hermetically sealed. The product may be prepared at and/or subsequently treated below room temperature (i.e., <20° C.), at room temperature (e.g., (approximately) 20° C.), or at increased temperature (>20° C., such as exemplarily at 60-120° C. for faster dissolving). The product may optionally be sterilized (e.g., by pasteurization, heating, irradiation with ultraviolet (UV) light or X-rays, gamma-rays, etc.).

In a preferred embodiment, the method further comprises a step of:
(a) fermenting the comestible nutrient composition;
(b) smoking the comestible nutrient composition;
(c) pickling a comestible good with a composition comprising the comestible nutrient composition, preferably in combination with one or more further component such as at least 1% by weight, referred to the composition, of sodium chloride, at least 1% by weight, referred to the composition, of one or more types of sugar, at least 1% by weight, referred to the composition, of acetic acid, at least 0.5% by weight, referred to the composition, of ethanol, or at least 5% by weight, referred to the composition, one or more types of edible oil; and/or
(d) thickening the comestible nutrient composition to obtain a syrup, in particular wherein sugar and/or other sweetener is added to obtain a sweet syrup.

As used herein, fermenting may be understood in the broadest sense as any kind of a metabolic process that produces chemical changes in the organic substrates as contained in the composition through the action of enzymes. Such enzymes may optionally originate from the cell-derived material, may be added and/or may be present in the form of microorganisms and/or secreted from such. In one embodiment, fermenting may be used for increasing umami taste. In one embodiment, fermenting may be used for increasing shelf-life. In one embodiment, a fermented comestible nutrient composition may, after the step of fermenting, comprise at least 0.5% by weight, at least 1% by weight, at least 2% by weight, at least 5% by weight, at least 10% by weight, based on the total weight of the composition, of cell-derived material.

In a preferred embodiment, the comestible nutrient composition comprises considerable contents of dead material derived from the cells of interest. In a preferred embodiment, the comestible nutrient composition comprises a mass ratio of the dead material derived from the cells of interest:viable cells of interest of at least 2:1, of at least 3:1, of at least 4:1, of at least 5:1, of at least 10:1, of at least 50:1, of at least 100:1, or dead material derived from the cells (essentially) free of viable cells.

A step of thickening may be conducted by any means. It may comprise partial removal of water and/or addition of further ingredient having thickening properties such as, e.g. one or more sugars, one or more other (poly)saccharides (e.g. starch, resistant starch, agar, pectin, inulin, fructans, raffinose, polydextrose), one or more thickening sweeteners, one or more thickening agents (e.g., cellulose, hemicellulose, carboxymethyl cellulose, carboxy ethyl cellulose, carboxypropyl cellulose, chitin, beta-glucan, raw guar gum, xanthan gum, lignin, a polyuronide, alginic acid or a salt thereof (e.g., sodium alginate), or a combination of two or more thereof).

Thickening the comestible nutrient composition may improve its properties to be swallowed and may prevent aspiration when adjusted to a desired degree (cf., Nishinary et al., npj Science of Food, 2019, 3:5). Mixing with a thickening agent (such as, e.g. sodium alginate) may optionally also be used to improve solubility of proteins (such as, e.g., myofibrillar proteins) (cf., Li et al., Food Biophysics, 2020, 15:113-121). Optionally thickening soluble aggregates of myofibrillar proteins may also be obtained by admixing gallic acid and heating (cf., Chen et al., J. Agric. Food Chem., 2020, 68:11535-11544). In contrast to these methods, in a preferred embodiment of the present invention, myofibrillar proteins are not purified or separated from other proteins or components of cultivated cells. This allows obtaining the full nutrient content of the cells.

In the context of the present invention, dead material derived from the cells of interest may be understood as material that was originally content of the cells of interest harvested in step (ii) (which may optionally be not present in viable cells any more). In the context of the present invention, cell viability may be understood as the ability to proliferate under suitable conditions.

In a preferred embodiment, the comestible nutrient composition is:
(a) a drinkable composition, in particular wherein the drinkable composition contains a mass ratio of dead material derived from the cells of interest:viable cells of interest of at least 2:1 or higher;
(b) a powder or granulate which is suspendable in aqueous solutions and water, in particular wherein the powder or a granulate composition contains a mass ratio of dead material derived from the cells of interest:viable cells of interest of at least 2:1 or higher; or
(c) a liquid or pasty composition that contains cell culture medium and living and/or dead cells, preferably wherein the cell-derived material derived from the step (ii) contains cell culture medium in which the cells were cultivated in the preceding step (i), in particular wherein the liquid or pasty composition is pasteurized.

In an alternative embodiment, the cells of interest remain (essentially) viable in the product of interest. For instance, >50% or >75% of cells, based on total cells harvested in step (ii) may remain viable.

### Optional Step (iv) of Admixing One or More Further Consumable Ingredients

As indicated above, the comestible nutrient composition may optionally further comprise one or more further consumable ingredients selected from the group consisting of one or more vitamins, one or more minerals, one or more aroma compounds, one more food colors, one or more types of fibers, ethanol, acetic acid, carbonic acid, and combinations of two or more thereof. Ingredients may interchangeably also designated as components, additives, etc.

This may allow compensating for common micronutrient deficiencies of consumers (e.g., iron, vitamin A and iodine, but possibly also zinc, folate, vitamin B12, other B vitamins, vitamin C, vitamin D, calcium, selenium and fluoride). In one embodiment, this may allow compensating micronutrient deficiencies of elderly consumers. In one embodiment, this may allow compensating micronutrient deficiencies of consumers having problems to chew and/or swallow and potentially even suffering from dysphagia. Thus, consumers having problems to chew and/or swallow bulk meat may be efficiently supplied by nutrients. The comestible nutrient composition may optionally also be used for enteral nutrition (e.g., nasogastric nutrition such as nasogastric intubation, gastrostomy, jejunostomy, or other type of tube feeding).

A vitamin may be any vitamin known in the art. For instance, vitamins that may be added are selected from the group consisting of vitamin A, vitamins B (e.g., vitamin B1, vitamin B2, Vitamin B3, vitamin B5, vitamin B6, folate, vitamin B12), vitamin C, vitamin D, and metabolic precursors and combinations of two or more thereof. A vitamin or metabolic precursor thereof may be of natural or synthetical origin (nature identical in structure or artificial in structure). It may be commercially available. Preferably, the comestible nutrient composition comprises not more than 20% by weight, not more than 10% by weight, not more than 5% by weight, not more than 2% by weight, or not more than 1% by weight, referred to the total mass of the comestible nutrient composition, of total amount of vitamins.

A mineral may be any nutrient that may be of nutrient value for consumers. Typically and preferably, minerals comprise ions or complexes of metals such as, e.g., a metals selected from the group consisting of iron, copper, calcium, zinc, and a combination of two or more thereof. Furthermore, minerals may also be selenium, iodine, and/or fluoride. A mineral may be commercially available. Preferably, the comestible nutrient composition comprises not more than 20% by weight, not more than 10% by weight, not more than 5% by weight, not more than 2% by weight, or not more than 1% by weight, referred to the total mass of the comestible nutrient composition, of total amount of minerals.

An aroma compound may be any comestible compound known in the art that alters the flavor or taste. Thus, the flavor or taste is rather freely selectable and may optionally also include non-meat-like flavors and even exotic flavors. Preferably, an aroma compound has a significant impact on the comestible nutrient composition's taste, when it is applied in low amounts. Preferably, the comestible nutrient composition comprises not more than 20% by weight, not more than 10% by weight, not more than 5% by weight, not more than 2% by weight, or not more than 1% by weight, referred to the total mass of the comestible nutrient composition, of total amount of aroma compounds. An aroma compound may be of natural or synthetical origin (nature identical in structure or artificial in structure). For example, an aroma compound may be menthol furaneolhexyl cinnamaldehyde, isovaleraldehyde anisic aldehyde cuminaldehyde, glutamate, fructone, ethyl methylphenylglycidate, dihydrojasmone, oct-1-en-3-one, 2-acetyl-1-pyrroline, 6-acetyl-2,3,4,5-tetrahydropyridine, delta-octalactone, massoia lactone, diacetyl acetoin, nerolin, and combinations of two more thereof. Many aroma compounds are known by those skilled in the art and may be commercially available.

A food color may be any comestible dye known in the art that alters the color of food, also designatable as food coloring. Preferably, a food colors has a significant impact on the comestible nutrient composition's color, when it is applied in low amounts. The coloring may be freely selectable and may, optionally, also be non-meat-like.

The comestible nutrient composition may have any texture, which may be defined by optionally added to the comestible nutrient composition. For example, as texturizing and/or nutrient, one or more polypeptides of extracellular matrix (e.g., collagen, elastin, etc.) may be added to the comestible nutrient composition. Nutrient value may be comparable to meat or even superior to meat.

Preferably, the comestible nutrient composition comprises not more than 20% by weight, not more than 10% by weight, not more than 5% by weight, not more than 2% by weight, or not more than 1% by weight, referred to the total mass of the comestible nutrient composition, of total amount of food colors. A food color may be of natural or synthetical origin (nature identical in structure or artificial in structure). For example, a food color may be beetroot juice, beta-carotene, quinoline yellow, Ponceau 4R, Patent blue V, Green S, Brilliant blue FCF, Citrus red 2, Orange B Indigotine, Fast green FCF, Erythrosine, Allura red AC, Tartrazine, Sunset yellow FCF, or a combination of two or more thereof. A food color may even be fluorescent such as fluorescein. Many food colors are known by those skilled in the art and may be commercially available.

Fibers may be any comestible fibers, which may also be designated as dietary fibers. Typically, fibers in the context of the present invention are plant-derived food ingredients that cannot be completely broken down by human digestive enzymes. For example, it may originate from legumes, whole grains and cereals, vegetables, fruits, nuts or seeds. For example, fibers may comprise or consist of cellulose, hemicellulose, chitin, pectin, resistant starch, or a combination of two or more thereof. Fibers may also comprise inulin, beta-glucan, raw guar gum, xanthan gum, fructans, lignin, a polyuronide, an alginic acid or a salt thereof (e.g., sodium alginate), agar, carrageen, raffinose, polydextrose, or a combination of two or more thereof. Many fibers are known by those skilled in the art and may be commercially available.

Based on the production process of the present invention, but also as component of the admixed finished comestible, proteins of the extracellular matrix (ECM) may be comprised in the composition, such as, e.g., collagen type I, hyaluronic acid, pyolysin, vinculin, laminin, fibronectin.

The comestible nutrient composition may optionally further comprise one or more ingredients as described in Malone, 2005 (“Enteral Formula Selection: A Review of Selected Product Categories”, in Nutrition Issues in Gastroenterology, Series #28, ed. Carol Rees Parrish, Practical Gastroenterology). The content ranges may optionally also as those taught in Malone, 2005, while preferably the milk-derived and/or plant-derived protein sources are replaced by the cell-derived material as (preferably main or sole) protein source. Preferably, the comestible nutrient composition comprises the cell-derived material as protein source and preferably does not comprise milk-derived and/or plant-derived protein sources, in particular does (essentially) not comprise milk-derived and does (essentially) not comprise plant-derived protein sources.

In one embodiment, the comestible nutrient composition may comprise ethanol, optionally in addition to one or more of the further ingredients mentioned herein. In certain embodiments of the present invention, it may comprise 0 to 80% by weight, 0 to 40% by weight, 0 to 6% by weight, 1 to 15% by weight, 5 to 15% by weight or 10 to 40% by weight, referred to the total mass of the comestible nutrient composition, of total amount of ethanol.

In one embodiment, the comestible nutrient composition may comprise acetic acid, optionally in addition to one or more of the further ingredients mentioned herein. In certain embodiments of the present invention, it may comprise 0 to 20% by weight, 0 to 6% by weight, 1 to 15% by weight, 5 to 15% by weight or 10 to 20% by weight, referred to the total mass of the comestible nutrient composition, of total amount of acetic acid.

In one embodiment, the comestible nutrient composition may comprise carbonic acid, optionally in addition to one or more of the further ingredients mentioned herein. In certain embodiments of the present invention, it may comprise 0 to 5% by weight, 0.1 to 2% by weight, 0.2 to 1% by weight, 0.3 to 0.8% by weight or 0.4 to 0.7% by weight, referred to the total mass of the comestible nutrient composition, of total amount of carbonic acid. These contents may refer to a fresh product, preferably provided in an (essentially) sealed container (e.g., bottle). The presence of carbonic acid may provide a sparkling drink. In one embodiment, the comestible nutrient composition may be fermented in such container (e.g., a bottle), i.e., the container may optionally act as a fermenter.

Optionally, the comestible nutrient composition may comprise one or more sugars, optionally in addition to one or more of the further ingredients mentioned herein. For instance, it may comprise one or more saccharides such as, e.g., saccharose, glucose, fructose, trehalose, sucrose, sorbitol, dextran, etc. This may improve the solubility of the cell-derived material at different pH values and/or may improve taste. For instance, the comestible nutrient composition may comprise 0 to 80% by weight, 0 to 40% by weight, 0 to 6% by weight, 1 to 15% by weight, 5 to 15% by weight or 10 to 40% by weight, referred to the total mass of the comestible nutrient composition, of total amount of sugars.

Optionally, the comestible nutrient composition may comprise one or more amino acids, optionally in addition to one or more of the further ingredients mentioned herein. For instance, it may comprise one or more natural amino acids. This may supplement a consumer in need thereof. For instance, the comestible nutrient composition may comprise 0 to 80% by weight, 0 to 40% by weight, 0 to 6% by weight, 1 to 15% by weight, 5 to 15% by weight or 10 to 40% by weight, referred to the total mass of the comestible nutrient composition, of total amount of amino acids.

Optionally, the comestible nutrient composition may comprise one or more polypeptides (also: proteins), optionally in addition to one or more of the further ingredients mentioned herein. For instance, it may comprise one or more polypeptides of natural origin (e.g., obtained from milk, bacteria, plant-based expression systems etc.). This may supplement a consumer in need of amino acids. For instance, the comestible nutrient composition may comprise 0 to 80% by weight, 0 to 40% by weight, 0 to 6% by weight, 1 to 15% by weight, 5 to 15% by weight or 10 to 40% by weight, referred to the total mass of the comestible nutrient composition, of total amount of polypeptides.

Optionally, the comestible nutrient composition may comprise one or more fats (also: lipids) and/or fatty acids, optionally in addition to one or more of the further ingredients mentioned herein. For instance, it may comprise one or more fats and/or fatty acids of natural origin (e.g., obtained from plants, fish and/or milk). This may contain essential fatty acids. This may contain unsaturated or multiple unsaturated fatty acids. This may supplement a consumer in need of fatty acids, in particular essential fatty acids. For instance, the comestible nutrient composition may comprise 0 to 80% by weight, 0 to 40% by weight, 0 to 6% by weight, 1 to 15% by weight, 5 to 15% by weight or 10 to 40% by weight, referred to the total mass of the comestible nutrient composition, of total amount of fats and/or fatty acids.

Optionally, the comestible nutrient composition may comprise electrolytes, optionally in addition to one or more of the further ingredients mentioned herein. Its osmolarity may hypotonic, isotonic or hypertonic compared to human blood (herein understood as the osmolarity of an aqueous saline consisting of water and 0.9% by weight sodium chloride). The osmolarity may be adapted and adjusted by the total content of dissolved ingredients in the comestible nutrient composition.

Optionally, the comestible nutrient composition may comprise pharmaceutically active ingredients such as, e.g., an adaptogen, a growth factor, a polyphenol, an antibody or antigen-binding fragment thereof, a small molecule drug, a prebiotic, and/or a nucleic acid (e.g., ribonucleic acid (RNA) (e.g., siRNA), deoxyribonucleic acid (DNA)), optionally in addition to one or more of the further ingredients mentioned herein.

In one embodiment, the comestible nutrient composition may also be suitable for enteral administration, such as enteral nutrition (e.g., nasogastric nutrition such as nasogastric intubation, gastrostomy, jejunostomy, or other type of tube feeding).

In one alternative embodiment, the comestible nutrient composition may also be suitable for parenteral administration, optionally in addition to one or more of the further ingredients mentioned herein. In one embodiment, the comestible nutrient composition may also be suitable for intravenous injection/infusion. In this case, it may be filtered. It may, optionally be suitable for treating either cellular dehydration or hypervolemia.

Optionally, the comestible nutrient composition may comprise one or more juices, optionally in addition to one or more of the further ingredients mentioned herein. For instance, it may comprise one or more fruit or vegetable juices.

The pH of the comestible nutrient composition may be adjusted to a desired range. Preferably, the pH is essentially neutral or sour. In one embodiment, the pH is in the range of pH 2-9, pH 2-8, pH 3-7, pH 4-7, pH 5-7, or pH 4-6. The pH may be adjusted by comestible acids, bases, buffer agents and/or acidity regulators.

In a preferred embodiment, a high-grade nutritional liquid product for individual demands may be obtained. The comestible nutrient composition may be adjusted to individual lifestyle demands, thus, its content ranges may be adapted to the desire of a specific consumer or group of consumers. This may be considered as “customizable food” and/or “personalized food”.

The comestible nutrient composition may be a cell-based drink. It may also be considered as “animal drink” or “cell drink”. It may also be understood as “clean meat”, i.e., nutrition of animal-like sources not obtained as meat from a slaughtered animal.

In a preferred embodiment, the step (iv) includes adding:
at least 0.01% by weight, referred to the whole comestible nutrient composition, at least one food color;
at least 0.2% by weight, referred to the whole comestible nutrient composition, carbonic acid to obtain a sparkling drink;
at least 5% by volume, referred to the whole comestible nutrient composition, of ethanol;
at least 0.01% by weight, referred to the whole comestible nutrient composition, at least one pharmaceutically active ingredient; or
a combination of two or more thereof.

In one embodiment, the step (iv) includes adding at least 0.01% by weight, at least 0.02% by weight, at least 0.05% by weight, at least 0.1%, or at least 0.5% by weight by weight, referred to the whole comestible nutrient composition, at least one food color. Preferably, the step (iv) does not include more than 5% by weight, more than 2% by weight, or more than 1% by weight, referred to the whole comestible nutrient composition, a total content of food colors.

In one embodiment, the step (iv) includes adding at least 0.1% by weight, at least 0.2% by weight, at least 0.3% by weight, 0.1 to 2% by weight, 0.2 to 1% by weight, 0.3 to 0.8% by weight or 0.4 to 0.7% by weight, referred to the whole comestible nutrient composition, or carbonic acid. This may provide a sparkling drink.

In one embodiment, the step (iv) includes adding at least 3% by volume, referred to the whole comestible nutrient composition, of ethanol. In one embodiment, the step (iv) includes adding at least 5% by volume, referred to the whole comestible nutrient composition, of ethanol. It may comprise adding 5 to 15% by weight or 10 to 40% by weight, referred to the total mass of the comestible nutrient composition, of total amount of ethanol.

In one embodiment, the step (iv) includes adding at least 0.01% by weight, at least 0.02% by weight, at least 0.1% by weight, or at least 0.5% by weight, referred to the whole comestible nutrient composition, at least one pharmaceutically active ingredient. A pharmaceutically active ingredients may, for instance, be an adaptogen, a growth factor, a polyphenol, an antibody or antigen-binding fragment thereof, a small molecule drug, a prebiotic, or a nucleic acid (e.g., ribonucleic acid (RNA) (e.g., siRNA), deoxyribonucleic acid (DNA)).

Optionally, the comestible nutrient composition may be hermetically sealed. For instance, it may be packaged in a container such as a bottle, closed glass, or a sealed beaker or Liquid packaging board. As far as it is a powder or granulate, it may also be packaged in a cardboard box and/or sealed by a film.

It will be understood that also the comestible nutrient composition obtainable (or obtained) from a method of the present invention bears particularly beneficial technical properties.

Accordingly, a further aspect of the present invention refers to a comestible nutrient composition obtainable (or obtained) from a method of the present invention.

It will be understood that the definitions and embodiments made in the context of the method of the present invention mutatis mutandis apply to the comestible nutrient composition obtainable from such method.

As indicated above, the comestible nutrient composition may have any form. In a preferred embodiment, the comestible nutrient composition is:
(a) a drinkable composition;
(b) a powder or granulate composition;
(c) a gel; or
(d) a frozen or partly frozen composition,
wherein the comestible nutrient composition may optionally form part of a filling of a capsule of may optionally form part of a drink, a dairy product, a non-dairy cream, a sauce, or a bakery good.

A drinkable composition may be in liquid or pasty form, preferably in liquid form.

As used herein, the term “gel” may be understood in the broadest sense and may also comprise a gel as such, any kind of jelly composition, a filling of a capsule, filling of a bakery good, a pudding form, etc.

The comestible nutrient composition may also be frozen or partly frozen composition, including an ice cube, frozen yogurt-like, soft ice like, slushies like.

A further aspect of the present invention refers to the use of a comestible nutrient composition of the present invention for providing a well-defined nutrient composition, in particular a well-defined protein composition, to a consumer.

It will be understood that the definitions and embodiments made in the context of the method and the comestible nutrient composition obtainable from such method of the present invention mutatis mutandis apply to the use thereof. The administration to a well-defined protein composition to a consumer may optionally be personalized nutrition. Thus, its nutrient composition may be optimized for an individual.

The comestible nutrient composition of the present invention is useful for being applied to patients and/or elderly persons. In particular, the comestible nutrient composition of the present invention is particularly useful for being applied as a drink to patients and/or elderly persons. This allows also consumption by persons who have problems to chew and/or to swallow. The present invention also refers to use of a comestible nutrient composition of the present invention for enteral nutrition (e.g., nasogastric nutrition such as nasogastric intubation, gastrostomy, jejunostomy, or other type of tube feeding).

As noted above, many subjects have intolerance towards soy, wheat or milk products that represent the key sources of proteins in protein compositions known in the art. The comestible nutrient composition of the present invention enables to avoid such drawbacks. Thus, the present invention also refers to use of a comestible nutrient composition of the present invention for nutrition of a subject who has an intolerance towards soy, wheat or milk products. The present invention also refers to use of a comestible nutrient composition of the present invention for enteral nutrition of a subject who has an intolerance towards soy, wheat or milk products

The comestible nutrient composition of the present invention may be an alternative for subjects, who cannot or can only hardly digest meat and/or proteins obtained from milk, egg, one or more plant sources (e.g. soy beans), one or more bacterial sources, fish, and/or similar sources or combinations thereof, but require optimal, nutrition, on particular personalized nutrition.

The comestible nutrient composition may be used to provide patients suffering from swallow problems with sufficient nutrition (e.g., dysphagia, as noted as a pathologic situation in the International Statistical Classification of Diseases and Related Health Problems (ICD) No. 10 (ICD-10) as R13. Accordingly, the present invention also refers to a comestible nutrient composition of the present invention for use in a method for treating a patient suffering from dysphagia with a well-defined nutrient composition. In other words, the present invention also refers to a method for treating a patient suffering from dysphagia with a well-defined nutrient composition, wherein said patient is administered with a sufficient amount of a comestible nutrient composition of the present invention. Preferably, the patient is administered with the comestible nutrient composition orally.

A further aspect refers to the comestible nutrient composition of the present invention for use in a method of treating a patient suffering from dysphagia, wherein the patient is preferably administered with the comestible nutrient composition by enteral nutrition (e.g., nasogastric nutrition such as nasogastric intubation, gastrostomy, jejunostomy, or other type of tube feeding), by swallowing a drinkable comestible nutrient composition, or a combination thereof.

It will be understood that the definitions and embodiments made in the context of the method and the comestible nutrient composition obtainable from such method of the present invention mutatis mutandis apply to the medical use thereof.

In other words, the present invention also relates to a method of treating a patient suffering from dysphagia, wherein the patient is administered with sufficient amounts of the comestible nutrient composition by enteral nutrition (e.g., nasogastric nutrition such as nasogastric intubation, gastrostomy, jejunostomy, or other type of tube feeding), by swallowing a drinkable comestible nutrient composition, or a combination thereof.

The comestible nutrient composition may be also consumed by gastric fistula patients, surgically reduced stomach volume, reduced ability to chew and swallow (including with dental prosthesis).

The comestible nutrient composition may be also consumed as a lifestyle product. Thus, the present invention also refers to the use of the comestible nutrient composition as lifestyle product.

The following examples illustrate the invention further. The scope of the invention is defined by the claims.

### FIGURES

FIG. 1 shows the detachment of pig muscle cells CD29++ Bio 8.1, passages 9/10 from collagen-coated 6-well plates using different reagents. FIG. 1A shows the non-detached, thus attached cells (t=0 min). FIG. 1B shows the cells after treatment with PBS with Ca²⁺ and Mg²⁺ (t=15 min). FIG. 1C shows the cells after treatment with PBS without Ca²⁺ and Mg²⁺ (t=15 min). FIG. 1D shows the cells after treatment with 1 mM sodium citrate in 270 mOsm/L PBS without Ca²⁺ and Mg²⁺ (t=15 min). FIG. 1E shows the cells after treatment with 1 mM sodium citrate in 570 mOsm/L PBS without Ca²⁺ and Mg²⁺ (t=15 min). FIG. 1F shows the cells after treatment with TrypLE (t=15 min). The scale bar denotes 100 μm.

FIG. 2 shows the attachment of CD29++ pig muscle cells (CD29++ Bio 8.1 passage 14 (P14)) on Cytodex3 microcarrier. Cells cultured in surface-repellent 6-well plates seeded with a cell to bead ratio of 13 using Cytodex3 microcarrier. Culture medium DMEM/F-12+20% FBS. FIG. 2A shows the cells before incubation (t=0 h). FIG. 2B shows the cells after incubation at 37° C., 5% CO₂ (t=28 h). FIG. 2C shows the cells after incubation at 37° C., 5% CO₂ (t=48 h). The scale bar denotes 100 μm.

FIG. 3 shows the attachment of pig muscle cells CD29++ Bio 8.1 P14 after 48 h of incubation at 37° C., 5% CO₂. Different cell/bead ratios (C/B) are compared with each other and the cells were stained nuclei using DAPI. Cultured in surface-repellent 6-well plates using Cytodex3 microcarrier were used. The culture medium was DMEM/F-12+20% FBS. FIG. 3A shows the cells, when a cell/bead ratio of 3 is used. FIG. 3B shows the cells, when a cell/bead ratio of 7 is used. FIG. 3C shows the cells, when a cell/bead ratio of 13 is used. The scale bar denotes 100 μm.

FIG. 4 shows at different cell/bead ratios (C/B) and different detachment solutions. The cells were cultured in surface-repellent 6-well plates using Cytodex3 microcarrier. The culture medium was DMEM/F-12+20% FBS. FIG. 4A shows pig muscle cells CD29++ Bio 8.1 P14 after 48 h of incubation at 37° C. before detachment (C/B ˜7). FIG. 4B shows pig muscle cells CD29++ Bio 8.1 P14 after 48 h of incubation at 37° C. and detachment using 15 mM sodium citrate in 570 mOsm/L PBS without Ca²⁺ and Mg²⁺ (C/B ˜7) before resuspending. FIG. 4C shows pig muscle cells CD29++ Bio 8.1 P14 after detachment procedure using 15 mM sodium citrate in 570 mOsm/L PBS without Ca²⁺ and Mg²⁺ after resuspending (C/B ˜7). FIG. 4D shows pig muscle cells CD29++ Bio 8.1 P14 after detachment procedure using 1 mM sodium citrate in 570 mOsm/L PBS without Ca²⁺ and Mg²⁺ after resuspending (C/B ˜8). FIG. 4E shows pig muscle cells CD29++ Bio 8.1 P14 after detachment procedure using only PBS without Ca²⁺ and Mg²⁺ after resuspending (C/B ˜13). FIG. 4F shows pig muscle cells CD29++ Bio 8.1 P14 after detachment procedure using 1 mM sodium citrate in PBS without Ca²⁺ and Mg²⁺ after resuspending (C/B ˜10). The scale bar denotes 100 μm.

FIG. 5 shows dose response curves for HepG2 cells with Doxorubicin (FIG. 5A, IC50=0.079 μM, n=3/SD, R²=0.9483, Sy.x=8.153) Neutral Red Assay (FIG. 5B, IC50=0.116 μM, n=4/SD, R²=0.9660, Sy.x=7.001). CellTiter Glo 2.0.

FIG. 6 shows the linearity of CellTiterGlo 2.0 with increasing amounts of cell lysate, which is dependent on amount of cell lysate and high HepG2 cell number (FIG. 6A, n=3/SD) and low HepG2 cell number (FIG. 6B, n=4/SD).

### EXAMPLES

### Example 1

### Skeletal Muscle Tissue Harvesting for Satellite Cell Isolation and Cryopreservation (Example of Mammalian Cells)

### 1. Tissue Harvesting (TCB)

Skeletal muscle tissue, ca. 2 g piece, is harvested from a ruminant (e.g., cow, sheep, goat, deer, buffalo, etc.) or a non-ruminant (e.g., pig etc.) or a pseudo-ruminant (e.g., horse, camel, rabbit, etc.). Once washed with preservation medium (phosphate buffered saline (PBS), supplemented with antibiotics on ice) in a 15 mL tube the material is transferred to newly prepared 10 mL of preservation medium for further processing.

### 2. Tissue Processing/Digestion (CLS)

Each muscle tissue is dissected into smaller pieces (ca. 1-3 mm³) using sterile scalpels. This is followed by enzymatic digestion using collagenase I and dispase, rinsing steps with PBS supplemented with 50× dilution with Pen/Strep solution (twice the normal concentration, market available 100× solution: 5000 units/mL penicillin G and 5 mg/mL streptomycin), and plating into 6 well plates coated with collagen type I. Each well is triturated using a 1000 μL pipette, resulting in single cells and cellular cluster. To obtain single cell population, a 40 μm cell sorter can be employed instead. Cell suspensions are spun down for 300×g for 5 min. Supernatants are removed, the pellets are resuspended in 8 mL PBS supplemented with antibiotics (1% Pen/Strep solution) and pooled into a 15 mL tube. After centrifugation at 300×g for 3 min, the cell pellet is resuspended in 4.5 mL DMEM/F-12 optionally supplemented with 5% fetal bovine serum (FBS) and antibiotics and pipetted into three wells of 12 well plate (1.5 mL/well).

### 3. Cell Culture/Proliferation (CLS)

Heterogeneous cell populations from muscle tissue are cultivated under the condition at 37° C., 5% CO₂, 95% air, 85-95% humidity.

The surface of culture wear is coated with collagen type I for the initial attachment of the cell. Heterogeneous cell populations are either proliferate (dividing and increase in size) or differentiate or rest phase as a monolayer (attached on the surface) during the cultivation with DMEM/F-12 supplemented with 5% FBS and antibiotics.

### 4. Cell Purification/Satellite Cells

### 4.1. Cultivation of Adult Stem Cells

Cell purification of satellite cells can be obtained as described in Schmidt et al. (Cellular and Molecular Life Sciences, 2019, 76:2559-2570) or Hindi et al. (Science Signaling, 2013, 6(272):DOI: 10.1126/scisignal.2003832). The development from satellite cells via myoblasts and myocytes to myotubes can be monitored using muscle-specific biomarkers. These include Pax3 and Pax7 for satellite cells, MyoD and Myf5 for myoblasts. Myocytes can be detected by myogenin. Myotubes are positive for Myosin Heavy Chain (MyHC). Furthermore, mature myotubes are multinucleated which can be detected using nuclear-specific dyes like DAPI and Hoechst. Expression of Desmin, another muscle-specific marker, increases over the course of myogenesis and can be detected starting at the myoblast stage.

4.2. MACS (Magnetic-Activated Cell Sorting) with CD29 (Surface Marker)

Further processing via MACS can include a method as described in Choi et al. (Food Sci. Anim. Resour., 2020, 40(5):852-859). The CD29-positive satellite cells are sorted using a MACS Cell Separation System (Miltenyi Biotec, Bergisch Gladbach, Germany) to purify the population. When the cells reach 80-90% confluency, the cells are dissociated using either TrypLE™ Express (Gibco) or Accutase (Innovative Cell Technologies, Inc.) or 1 mM hypertonic (adjusted with potassium chloride into 570 mOsm/kg final osmolality) sodium citrate solution (Nie et al., (PlosONE, 2014, 9(1):e88012). The dissociated satellite cells are reacted with an anti-CD29 antibody (MAB17783, Novus Biologicals) and anti-mouse IgG microbeads (1:5; Miltenyi Biotec). The CD29-positive cells are sorted on an MS column (capacity, 1×10⁷ magnetically labeled cells, Miltenyi Biotec) according to the manufacturer's instructions. The identification of the sorted CD29-positive cells as muscle stem cells is verified by immunostaining with CD29 and Pax7.

### 4.3. Ice-Cold Treatment

The cells are optionally subjected to an ice-cold treatment as described in Benedetti et al. (SkeletalMuscle, 2021, 11(7):doi.org/10.1186/si3395-021-00261-w). Satellite cells are more sensitive to cold and detach from the surface faster than any other cell populations in processed cell suspension. Harvested or cultivated heterogeneous cell populations are applied on uncoated T-25 flask and incubate at 37° C. for 1 h (Plating No. 1). The supernatant is collected, containing the non-adherent cells, into a new 15 mL tube. Fresh proliferation media (DMEM supplement with 10% FBS and antibiotics) is added to the flask containing the adherent cells, mostly fibroblasts and myogenic cells in late development stage such as myoblast and myocytes.

The Falcon tube is centrifuged at 180×g for 10 min, the supernatant is discarded and the pelleted cells are resuspended in 3 mL (for a T-25 or 6 mL for T-75) of proliferation medium. The plating procedure is repeated (Plating No. 2). The supernatant is collected, containing the non-adherent cells, into a new 15 mL Falcon tube. Fresh proliferation media is added to the flask containing the adherent cells. The 15 mL tube is centrifuged at 180×g for 10 min, the supernatant is discarded and the pelleted cells are resuspend in desired volume of proliferation medium. The resulting cells are plated in a 0.1% gelatin coated T-25 or T-75 flask and incubate at 37° C. for overnight. The next day, the flask is washed 3 times with 5 mL (for T-25 and 10 mL for T-75) of PBS. The 5 mL (for T-25) and 10 mL (for T-75) of ice-cold PBS is added to the flask. The flask is placed on ice (approximately 0° C.) for 30 min with occasional gentle manual shaking (swirling motion). The supernatant is collected, containing the detached cells, into a 15 mL tube and centrifuge at 180×g for 10 min. The supernatant is discarded and the cells are resuspended in 3 mL for T-25 or lower volume of proliferation medium. A 0.1% gelatin-coated vessel is seeded with the resuspended cells and incubated at 37° C. Ice-cold procedure (after plating #2) is optionally performed at each passaging.

### 5. Cell Preservation/Cryopreservation for the Cell Bank

### 5.1. With DMSO

Single cell suspension is collected from either 2D or 3D culture after the washing process with PBS and either enzymatic digestion (Collagenase) or chelate treatment (ethylenediaminetetraacetic acid (EDTA), sodium citrate). The suspension is centrifuged at 180×g for 5 min and the supernatant is discarded.

The pellet is resuspended with 10% (v/v) DMSO with 90% (v/v) culture medium and aliquot 1 mL/cryotube at 4° C. The resulting tube is stored at −80° C. (Mr. Frosty (Nalgene, Merck)). After ≥24 h, the tubes are transferred to a liquid N₂ tank. When using the cells, the cells are thawed from preserved state. The vials are kept to be thawed in the liquid N₂ in the dewar vessel. Thawing may be performed in pre-warmed (37° C.) water bath immediately after taking out from liquid N₂. When observing 80% of cryopreservation medium liquidized 1 mL of pre-warmed medium are added into cryotube. The cells may be further handled by gentle pipetting and adding 8 mL of basal medium (4° C. or on ice) in a 15 mL tube. The suspension is centrifuged (4° C.) and plated in a culture dish and culture in an incubator.

### 5.2. Without DMSO

Single cell suspension is collected from either 2D or 3D culture after the washing process with PBS and either enzymatic digestion (collagenase) or chelate treatment (EDTA, sodium citrate). The suspension is centrifuged at 180×g for 5 min and the supernatant is discarded. The cell pellet is resuspended with 200 μL of DMSO-free freezing medium, StemCell Keep (Abnova) for 5×10⁵-5×10⁶ cells. The cryotube is transferred into a liquid N₂ chamber (−196° C.). Thawing from preserved state is performed by keeping the vials to be thawed in the liquid N₂ in the dewar vessel. The pre-warmed (37° C.) medium (1 mL) is directly added to the vial immediately after taking out from liquid N₂. The solution is thawed by gentle pipetting and add into the 9 mL of basal medium (4° C. or on ice) in a 15 mL tube. The suspension is centrifuged (4° C.) and plated in a culture dish and culture in an incubator.

### Example 2

### Harvesting and Cultivation of Avian Muscle Cells

Other non-ruminants (e.g., poultry; chicken, turkey, duck, goose, fowl, pigeon, etc.) can also be used as an originate of cells. Skeletal muscle thereof are be harvested in same manner as described above. Avian cells are used for vaccine production (scalable in-suspension serum-free production of the vaccines). Culturing avian cells are comparably to the mammalian cell culture as mentioned above, except that cultivation temperature might slightly higher than mammalian cells because of their original body temperature (chicken: ca. 41-42° C., newly hatched chicken is ca. 40° C.).

### Example 3

### Harvesting and Cultivation of Non-Muscular Avian Cells

Avian (poultry) eggs and feather can be used as cell source. From chicken eggs (before hatch, ca. 21 days old chicken embryo) is harvested at any developmental stage such as embryonic stem cell, embryonic germ stem cell, progenitor cell, and fully developed/functioned cell. Chicken embryonic stem cells can proliferate unlimitedly and can be differentiated into any desired cell types. Cells and also be harvested from bone marrow. From bone marrow, high quantities of mesenchymal stem cells can be harvested. Mesenchymal stem cell can be differentiated into myocyte, adipocyte, osteoblast, neuron, and chondrocyte. Mesenchymal stem cells can secrete beneficial growth factors/cytokines for culturing cells as well. Cells can also be harvested from feather from poultry. Follicle cells can be harvested from feather. Follicle cells have similar characteristic like mesenchymal stem cell.

### Example 4

### Harvesting and Cultivation of Non-Muscular Avian Cells

The cells can be harvested from any organs/tissues of all biological kingdoms such as animal, plant, fungi, protista, eubacteria, and archaebacteria for cultivation to proliferate cells as well as obtain macro- and micro-nutrients.

### Example 5 Application Example

### Protein/Selenium Rich Cell-Based Composition (Water with Pork Myocyte- and Adipocyte-Based Solution)

The pork muscle's main component is myofibers, which are composed of muscle cells with white adipose tissues. In the case of the cells and extracellular matrices (ECMs) freshly harvested from a bioreactor, 10% (w/v) pork muscle cell contains (ca. 1 billion cells, 10 g) water-based 100 mL solution expect high protein (1.7 g, 3.4% DV (reference daily intake in percent, i.e., percent of suggested daily intake)) and high selenium (0.24 mg, 4.5% DV) with low carbohydrate amount (0 g). The daily recommendation of fluid consumption volume for healthy adult men is 3.7 L. When half of the volume 1.8 L fluid is replaced by water with pork myocyte-based solution, one can reach 30.6 g protein (61% DV) and 4.32 mg selenium (81% DV). The amount of saturated fatty acids (14.2 g/1.8 L, 70% DV) is adjustable.

### Example 6 Application Example

### Protein/Vitamin B12/D Rich Cell-Based Composition (Water with Salmon Myocyte- and Adipocyte-Based Solution)

When applied the same reverse calculation as Example 1, from 1.8 L of salmon cell-based drink, 36.9 g protein (73.8% DV), 4.74 mg vitamin B12 (311% DV), and 19.63 mg vitamin D (97.2% DV) are expectable.

### Example 7 Application Example

### Protein/Vitamin B12/Zinc/Iron Rich Cell-Based Composition (Water with Beef Myocyte- and Adipocyte-Based Solution)

From 1.8 L of beef cell-based drink, 35 g protein (70.2% DV), 3.55 mg vitamin B12 (147.6% DV), 8.19 mg zinc (73.8% DV), and 3.58 mg iron (19.8% DV) are expected. Upon mixing all the above-mentioned three examples (pork, salmon, and beef) in a 1:1:1 ratio 1.8 L cell-based drink would be 34.08 g protein (68.4% DV), 42.12 mg Selenium (76.8% DV), 4.092 mg vitamin B12 (170.4% DV), 6.54 mg vitamin D (32.4% DV), 4.284 mg zinc (39% DV), and 8 mg iron (10.8% DV). Supplementing this mixed cell-based drink with iron together with vitamin C, dietary fiber, and flavor may be consumed not only functional food, but also individual lifestyle demands. Additives may optionally be added such as, e.g, local crops, fruits, and vegetables to enhance/control the nutritional values.

### Example 8 Application Example: Liver Cell-Based Composition

Vitamin A, vitamin B12, copper-rich goose liver cell-based drink (1.8 L) contains 16.8 mg vitamin A (1861% DV), 97.2 μg vitamin B12 (4050% DV), 13.5 mg copper (1505% DV) is indicated that exceed more than 15 times higher DV (%) for a healthy adult man. Therefore, such liver cell-based drink may be consumed in lower amount of 120 mL (copper 100% DV) as a recommendation. The liver cells may be a valuable source of these nutritional supplements for other cell-based compositions.

### Example 9 Application Example

### Cell-Based Composition Comprising Different Types of Cells

A cell-based drink is prepared by admixing muscle cells from pork, salmon and beef in a mass ratio of 1:1:1. This is compared with comparable compositions comprising a comparable content of one cell type only. Obtainable nutrient contents are provided in the table below.

TABLE 1A
Nutrient contents in weight

| | Pork | Salmon | Beef | 1:1:1 Mix |
|---|---|---|---|---|
| Protein | 30.384 g | 36.9 g | 34.956 g | 34.08 g |
| Selenium | 44.28 mg | 56.52 mg | 25.56 mg | 42.12 mg |
| Vitamin B12 | 1.26 mg | 7.47 mg | 3.546 mg | 4.092 mg |
| Vitamin D | 0 mg | 19.62 mg | 0 mg | 6.54 mg |
| Zinc | 3.96 mg | 0.702 mg | 8.19 mg | 4.284 mg |
| Iron | 1.584 mg | 0.684 mg | 3.582 mg | 1.95 mg |

TABLE 1B
Nutrient contents in a 1.8 L of cell-based drink DV (%) (reference daily
intake in percent) and 1:1:1 mixed mass ratio DV (%)

| | Pork | Salmon | Beef | 1:1:1 Mix |
|---|---|---|---|---|
| Protein | 61.2 | 73.8 | 70.2 | 68.4 |
| Selenium | 81 | 102.6 | 46.8 | 76.8 |
| Vitamin B12 | 52.2 | 311.4 | 147.6 | 170.4 |
| Vitamin D | 0 | 97.2 | 0 | 32.4 |
| Zinc | 36 | 7.2 | 73.8 | 39 |
| Iron | 9 | 3.6 | 19.8 | 10.8 |

TABLE 2
Percentage amino acid (AA) contents (hydrolysis product) of pork muscle cells

| | | | mass of AA | AA in 10⁶ | mass of AA | Essential |
|---|---|---|---|---|---|---|
| | % of total | % AA of | in per 3 ng | myoblast | per 3 μg | amino acid |
| Amino | protein in | total cell | myoblast | suspension | muscle fibre | RDA adult |
| acid (AA) | muscle cell | weight | (ng) | (μg/ml) | (ng) | (mg/kg) |
| Thr | 3.09 | 0.71 | 0.021 | 0.02 | 21 | 20 |
| Val | 4.05 | 0.94 | 0.028 | 0.03 | 28 | 24 |
| Ile | 2.39 | 0.55 | 0.017 | 0.02 | 17 | 19 |
| Leu | 4.09 | 0.94 | 0.028 | 0.03 | 28 | 42 |
| Phe | 2.79 | 0.64 | 0.019 | 0.02 | 19 | 33 |
| Lys | 4.11 | 0.95 | 0.028 | 0.03 | 28 | 38 |
| Trp | 0.57 | 0.13 | 0.004 | 0.00 | 4 | 3.3 |
| Met | 1.62 | 0.37 | 0.011 | 0.01 | 11 | 14 |
| Arg | 3.38 | 0.78 | 0.023 | 0.02 | 23 | 31 |
| His | 3.17 | 0.73 | 0.022 | 0.02 | 22 | 10 |
| Asn | 1.52 | 0.35 | 0.011 | 0.01 | 11 | |
| Asp | 0.34 | 0.08 | 0.002 | 0.00 | 2 | |
| Ser | 3.71 | 0.86 | 0.026 | 0.03 | 26 | |
| Gln | 19.76 | 4.56 | 0.137 | 0.14 | 137 | |
| Glu | 4.6 | 1.06 | 0.032 | 0.03 | 32 | |
| Tyr | 2.36 | 0.55 | 0.016 | 0.02 | 16 | |
| Pro | 0.89 | 0.21 | 0.006 | 0.01 | 6 | |
| Gly | 7.7 | 1.78 | 0.053 | 0.05 | 53 | |
| Ala | 21.39 | 4.94 | 0.148 | 0.15 | 148 | |
| Cys | | 0.26 | 0.008 | 0.01 | 8 | |
| Rest | 8.47 | 1.96 | | | | |
| Total | 100 | 23.1 | | | | |
| protein | | | | | | |

### Example 10 Cultivating and Detachment of Cells

It was investigated how CD29++ sorted (twice with magnetic activated cell isolation with CD29 antibody, MACS) pig muscle cells detach from cell culture 6-well plates (collagen-coated and non-coated) using variations of phosphate buffered saline (PBS), e.g., with and without Ca²⁺, Mg²⁺, sodium citrate with isotonic or hypertonic osmolarities. Furthermore, CD29++ pig muscle cells were tested for attachment and detachment on Cytodex3 microcarrier using the before tested detachment solutions.

### 1. Materials and Methods

### 1.1. Reagents and Consumables

The used reagents and consumables for the conducted experiment are depicted below. All chemicals and reagents as well as the inoculation were prepared under the clean hood.
DMEM/F-12 w/o Glutamine, w/o Hepes (VWR, LOT/Article No. 392-0409P),
Glutamax™-1 (100×) (Life Technologies Corporation, LOT/Article No. 2390275),
fetal bovine serum (FBS) (Sigma, LOT/Article No. 0001650386),
TrypLE™ Express (1×) (TrypLE) (recombinant enzyme, Life Technologies Corp., LOT/Article No. 12604013),
PBS without (w/o) Mg²⁺, Ca² and phenol red (Hi-Media Laboratories Pvt. Ltd., LOT/Article No. TL1006),
PBS with Mg²⁺ and Ca² (PBS w/Mg²⁺, Ca²), without phenol red (Hi-Media Laboratories Pvt. Ltd., LOT/Article No. TL1023),
glucose mono hydrate (NeoLab, LOT/Article No. TC208-1 KG),
KCl (Biofroxx, LOT/Article No. 1617KG001),
tri-sodium citrate dihydrate (Sigma, LOT/Article No. W302600),
acridine orange propidium iodide stain (Aligned Genetics Inc., LOT/Article No. APOBAK1001),
6-well culture plate (neoLab, LOT/Article No. GF-0074),
collagen from calf skin (Sigma, LOT/Article No. C8919),
Cytodex3 microcarier beads usable as solid support (Cytiva, LOT/Article No. GEHE17_0485-02_P), and
double distilled water (H₂O_{dd}).

The PBS solutions can be iso-osmolar (ca. 270 Osm/L), or have an osmolarity of 570 mOsm/L. As far as not stated otherwise, PBS has an osmolarity of 270 Osm/L.

### 1.2. Further Equiment

CO₂ Incubator, microscope, Luna-fl™ Dual fluorescence cell counter

2. Preparation of Sodium Citrate Solutions with Different Osmolarities in PBS

The 1 mM hypertonic sodium citrate solution was prepared by dissolving 0.2941 g of tri-sodium citrate dihydrate and 11.487 g of KCl in PBS w/o Ca, Mg. The solution volume was adjusted to one liter with PBS. The osmolarity of the solution was estimated to be 570 mOsm/L according to Nie et al., (“Scalable Passaging of Adherent Human Pluripotent Stem Cells”, PLoS ONE, 2014, 9(1): e88012.doi:10.1371/journal.pone.0088012) with PBS instead of distilled water. The preparation of sodium citrate solutions with different osmolarities was performed by dissolving less amount of KCl in PBS with subtraction of the osmolarity provided by PBS (˜270 mOsm/L). The amount of sodium citrate was adjusted respectively.

### 3. Incubation of Pig Muscle Cells (CD29⁺⁺) in 6-Well Plates

For investigating the detachment of CD29⁺⁺ pig muscle cells using sodium citrate in PBS and other reagents, the cells were first incubated in 6-well plates (collagen-coated and non-coated). For this, CD29⁺⁺ pig muscle cells with passaging number 10 and 11 with a viability of 99.30% and 98.60% were used and wells were seeded with 15000 cells/cm². The wells were then filled up to 2 mL with DMEM/F-12+20% FBS and incubated at 37° C., 5% CO₂ for 72 h until 80-90% confluency was reached (incubation time depending on the seeding density).

Each detachment was performed in biological duplicates. The experimental setup using the 6-well plate including the detaching medium (R_{detach}) and the detaching time (t_{incubation}) was as follows:
1. R_{detach}=H₂O_{dd}, t_{incubation}=15 min;
2. R_{detach}=PBS with Ca²⁺ and Mg²⁺, t_{incubation}=15 min;
3. R_{detach}=PBS without Ca²⁺ and Mg²⁺, t_{incubation}=15 min;
4. R_{detach}=TrypLE, t_{incubation}=15 min;
5. R_{detach}=1 mM sodium citrate in 270 mOsm/L PBS, t_{incubation}=15 min; and
6. R_{detach}=1 mM sodium citrate in 570 mOsm/L PBS, t_{incubation}=15 min.
   4. Exemplary Detachment Procedure from 6-Well Plates

Detachment of CD29++ pig muscle cells in 6-well plates is conducted as follows:
(i) Aspirating cell culture medium;
(ii) Washing cells 2 times with 2 mL of the solution that is going to be used for detachment (wash with PBS without Ca²⁺ and Mg²⁺ for TrypLE treatment) (pre-warmed solutions to 37° C.);
(iii) Adding 2 mL of the respective detachment solution (0.5 mL for TrypLE) and incubating at 37° C., 5% CO₂ for 15 min. (detachment solutions with pH 7.2, pre-warmed to 37° C.);
(iv) Adding 2 mL of complete culture medium (DMEM/F-12+20% FBS)
(v) Resuspending/flushing cells from the surface by pipetting gently up and down (10 times) (optionally);
(vi) Taking pictures of detached cells/aggregates;
(vii) Transferring suspension with cells from 6-well plate into a 15 mL Falcon tube and centrifuge for 5 min at 300×g;
(viii) Removing supernatant and resuspending cells in ˜0.4 mL culture medium; and
(ix) Measuring cell viability with the Luna-fl™ Dual fluorescence cell counter using acridine orange/propidium iodide.

### 5. Cultivation of CD29⁺⁺ Bio 8.1 Pig Muscle Cells on Cytodex3 in 6-Well Plates

For investigating the attachment of CD29⁺⁺ Bio 8.1 pig muscle cells onto Cytodex3 microcarrier, pig muscle cells of passaging number 14 with a viability of 98.60% were seeded onto Cytodex3 microcarrier in a surface repellent 6-well plate. Seeding density was adjusted to get different cell to bead ratios as depicted in the following figure. The 6-well was then incubated for 48 h at 37° C. at 5% CO₂. DMEM/F-12+20% FBS was used as medium. Total volume of one well was 2 mL. Attachment of the cells was monitored by taking microscopic pictures.

The experimental setup for attachment of CD29⁺⁺ Bio 8.1 pig muscle cells on Cytodex3 microcarrier in a surface repellent 6-well plate is as laid out below. Different cell to bead ratios (Ratio_{Cell/Bead}) within a range of ˜3 to 13 are tested for attachment of the cells. Detachment of cells with the corresponding detachment reagents (detaching medium, R_{detach})) are as follows:
1. Ratio_{Cell/Bead}=13.33, R_{detach}=PBS without Ca²⁺ and Mg²⁺;
2. Ratio_{Cell/Bead}=10.00, R_{detach}=1 mM sodium citrate in PBS without Ca²⁺ and Mg²⁺;
2. Ratio_{Cell/Bead}=8.00, R_{detach}=1 mM sodium citrate in 570 mOsm/L PBS without Ca²⁺ and Mg²⁺;
3. Ratio_{Cell/Bead}=8.00, R_{detach}=1 mM sodium citrate in 570 mOsm/L PBS without Ca²⁺ and Mg²⁺;
4. Ratio_{Cell/Bead}=6.67, R_{detach}=15 mM sodium citrate in 570 mOsm/L PBS without Ca²⁺ and Mg²⁺;
5. Ratio_{Cell/Bead}=5.00, R_{detach}=hypotonic solution (8 g/L NaCl, 0.4 g KCl, 1 g/L glucose in H₂O_{dd} (in accordance with to Lai et al., “ESR studies on membrane fluidity of Chinese hamster ovary cells grown on microcarriers and in suspension”, Exp. Cell Res., 1980, 130:437-442); and
6. Ratio_{Cell/Bead}=3.00, R_{detach}=TrypLE.

Detachment of the cells from Cytodex3 was conducted as follows:
(i) Aspirating culture medium as good as possible by tilting the plate;
(ii) Washing cells with 1-2 mL PBS without Ca2+ and Mg²⁺;
(iii) Adding 2 mL of respective detachment reagent (pre-warmed to 37° C., pH 7.2)
(iv) Incubating at 37° C., 5% CO₂ for 15 min; and
(v) Shaking plate gently or flushing cells from microcarrier by pipetting up and down gently.

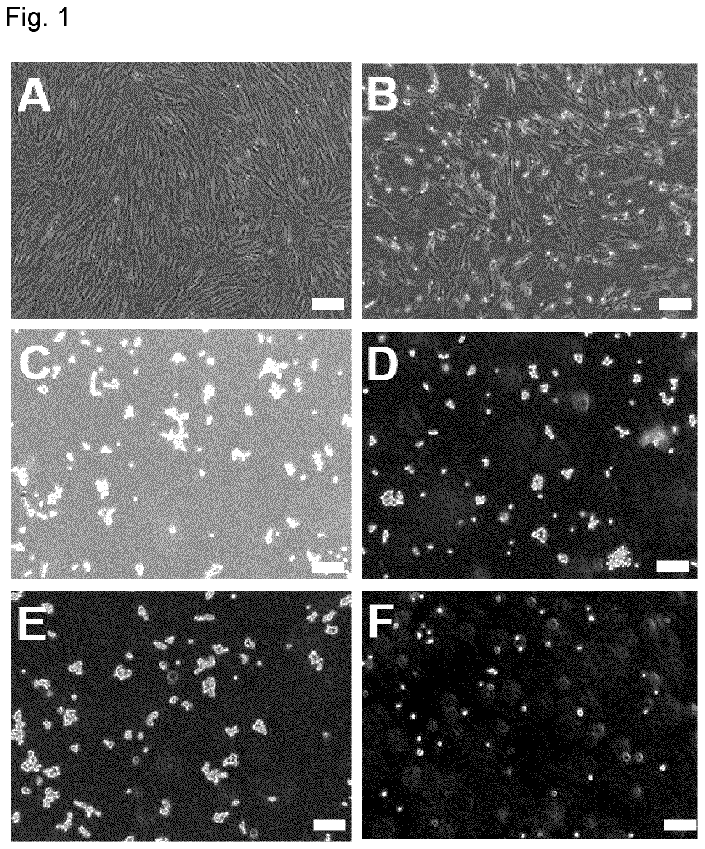

The results are depicted in FIG. 1. It is visible that the cells can be detached by means of PBS without Mg²⁺ and Ca²⁺, in particular when adding sodium citrate.

6. Viability of Detached Cells from Collagen Coated 6-Well Plate

The viability of the detached cells using the respective solutions of sodium citrate in PBS is depicted in the following Table 3.

TABLE 3
Viability of the detached cells, passage 10 (P10), using different concentrations
of sodium citrate in PBS (from collagen-coated 6-well plate). Herein
SD is the standard deviation and SC is sodium citrate.

| | | | | | 1 mM SC in | 1 mM SC in |
|---|---|---|---|---|---|---|
| | | | | | 270 mOsm/L | 570 mOsm/L |
| Sodium citrate | | PBS w/ | PBS w/o | | PBS w/o Ca, | PBS w/o Ca, |
| [mM] | H₂O_{dd} | Ca, Mg | Ca, Mg | TrypLE | Mg | Mg |
| MV Viability [%] | 0 | 89.70 | 93.00 | 88.35 | 93.55 | 95.80 |
| SD Viability [%] | 0 | 2.20 | 0.50 | 2.55 | 2.25 | 0.65 |
| MV Cell Count | 0 | 5.10E+04 | 1.06E+05 | 4.56E+05 | 1.59E+05 | 9.52E+04 |
| [1/mL] | | | | | | |
| SD Cell Count | 0 | 7.80E+03 | 3.18E+04 | 2.00E+04 | 7.20E+03 | 7.20E+03 |
| [1/mL] | | | | | | |

As a result, it is visible that the addition of a citrate salt such as sodium citrate can improve the detachment of cells.

Furthermore, the detachment of CD29++ pig muscle cells was tested on non-coated 6-well plates for comparison. For this, the usage of H₂O_{dd} was replaced by using 1 mM sodium citrate (SC) in tap water. Further, the standard deviation (SD) is depicted.

TABLE 4
Viability of detached cells, P11, using different concentrations
of sodium citrate in PBS (from non-collagen coated 6-well plate)

| | | | | | 1 mM SC in | 1 mM SC in |
|---|---|---|---|---|---|---|
| | 1 mM | | | | 270 mOsm/L | 570 mOsm/L |
| Sodium | SC in | PBS w/ | PBS w/o | | PBS w/o | PBS w/o |
| citrate [mM] | tap water | Ca, Mg | Ca, Mg | TrypLE | Ca, Mg | Ca, Mg |
| MV Viability [%] | 41.35 | 91.50 | 95.95 | 96.85 | 95.70 | 93.10 |
| SD Viability [%] | 2.05 | 5.30 | 1.45 | 0.45 | 0.70 | 0.80 |
| MV Cell Count | 8.66E+04 | 2.84E+04 | 9.24E+04 | 3.89E+05 | 1.19E+05 | 7.08E+04 |
| [1/mL] | | | | | | |
| SD Cell Count | 1.00E+03 | 2.58E+03 | 1.40E+04 | 5.50E+04 | 1.96E+04 | 8.80E+03 |
| [1/mL] | | | | | | |

As a result, it is visible that the addition of a citrate salt such as sodium citrate can improve the detachment of cells. Even the mere addition of a citrate salt such as sodium citrate can result in the significant detachment of cells. This is visible from the comparison of water and water with 1 mM sodium citrate (cf. first columns in Tables 3 and 4).

### 7. Attachment of CD29++ Pig Muscle Cells on Cytodex3 Microcarriers

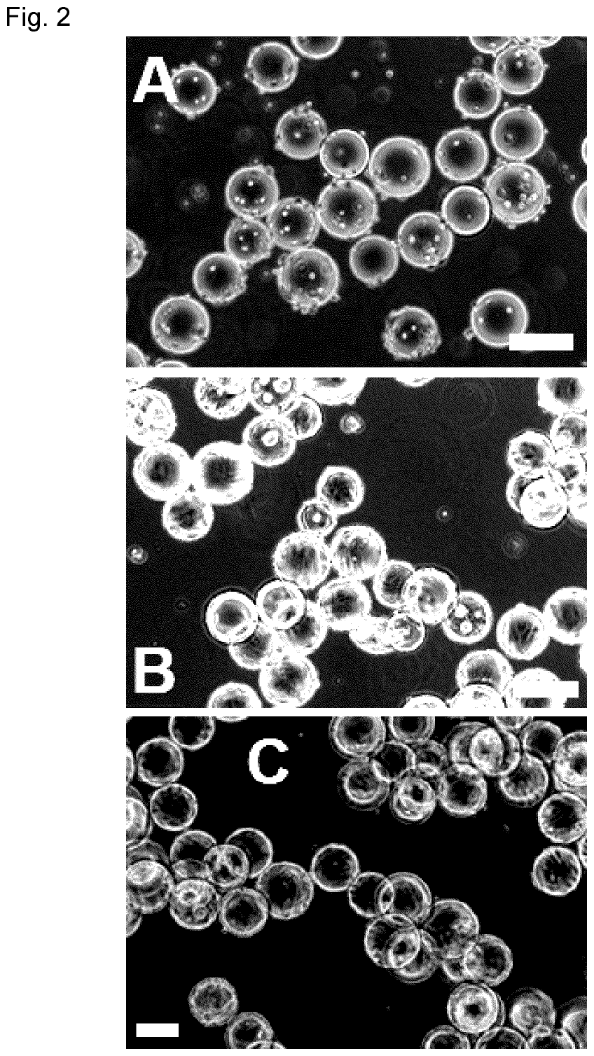

In a visualized experiment, CD29++ Bio 8.1 P14 cells were seeded of a Cytodex3 microcarrier. A cell/bead ratio of 13 was used. The results before, during and after incubation are depicted in FIG. 2. The cells were confluent on Cytodex3 microcarrier after 48 h of incubation using a cell/bead ratio of 13.

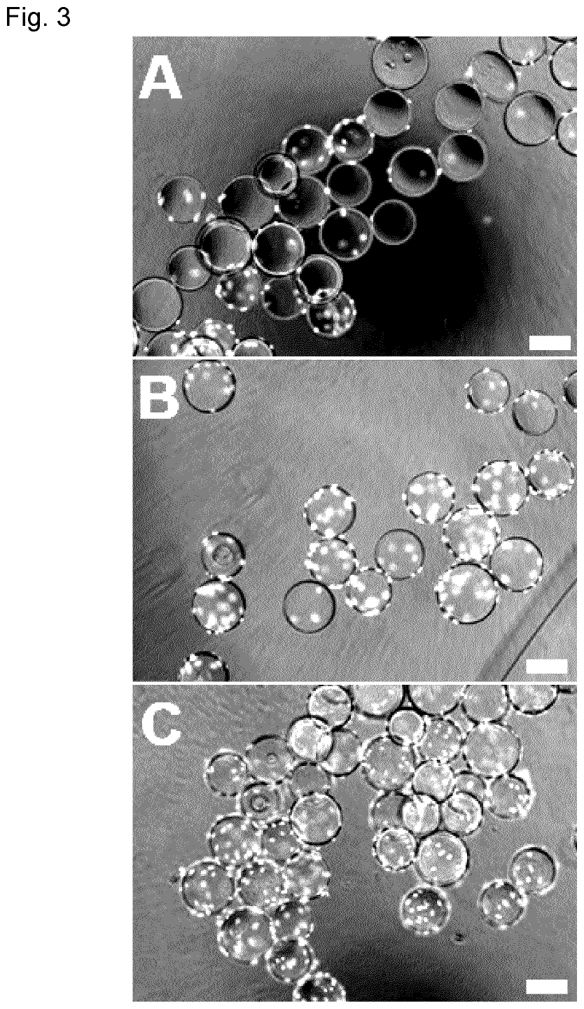

Furthermore, different cell/bead ratios were compared with each other. The results before, during and after incubation are depicted in FIG. 3. When staining cells using DAPI, the density of cells on microcarrier was visible the best for the cell/bead ratio of 13 (C/B 13).

8. Detachment of CD29++ Bio 8.1 Pig Muscle Cells from Cytodex3 Microcarriers

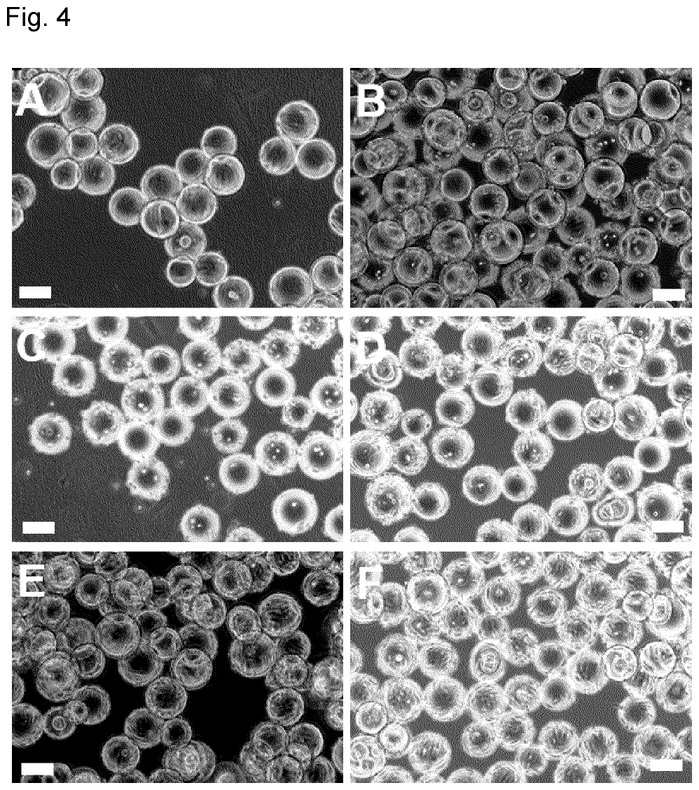

Detachment from Cytodex3 microcarrier was conducted for CD29++ Bio 8.1 P14 after 48 h of incubation at 37° C. The results are depicted in FIG. 4.

### 9. Conclusion

9.1. Detachment from Culture Plates

Detachment of cells (exemplified as CD29++ pig muscle cells, passages P10 and P11), using variations of buffers (exemplified on the basis of PBS buffers) showed, that the attachment of the cells appears to be probably Ca²⁺-ion dependent. In the examples, the highest viability could be reached using a citrate salt (here exemplified as 1 mM sodium citrate) in a hyperosmolar buffer (herein exemplified as 570 mOsm/L PBS) (here: 95.65%±2.15%). In contrast, 95.95%±1.45% viability was reached using only PBS without Ca²⁺ and Mg²⁺ when detaching from non-coated 6-well plate. In summary, no significant difference in detachment between collagen coated and non-collagen coated surfaces was observed. Without being bound to this theory, the differences in cell count are considered to be due to the fact that TrypLE detaches the cells from the surface as single cells, whereas sodium citrate and PBS or a combination of both (1 mM sodium citrate in PBS without Ca²⁺ and Mg²⁺ or 15 mM sodium citrate in PBS without Ca²⁺ and Mg²⁺ detaches the cells from the surface as larger aggregates. When comparing the microscopic images, it could be seen that essentially the entire surface of the 6-wells was detached from cells during detachment with only PBS or with the combination of sodium citrate and PBS without Ca²⁺ and Mg²⁺.

9.2. Detachment from Cytodex3 Microcarrier

Attachment of cells (exemplified as CD29++ Bio 8.1 pig muscle cells) was observed on a solid carrier (exemplified as Cytodex3 microcarrier). The well containing a cell to bead ratio ˜13 was confluent after 48 h of incubation at 37° C. and 5% CO₂. A suitable cell to bead ratio of 7 and upwards could be qualitatively determined for CD29++ Bio 8.1. Detachment of CD29++ Bio 8.1 worked using 15 mM sodium citrate in 570 mOsm/L PBS without Ca²⁺ and Mg²⁺. Detachment of CD29++ pig muscle cells worked from Cytodex3 microcarrier using a solution of 15 mM sodium citrate in 570 mOsm/L PBS without Ca²⁺ and Mg²⁺. It is expected to be scientifically reasonable that cells such as, e.g., CD29+ pig muscle cells, are also attachable and detachable from different microcarriers like Cytodex1 etc.

### Example 11 Cell Pulp Processing

The protein solubilization behaviour of lyophilized meat cell-containing material (exemplified as chicken meat cell-containing material) was conducted under different conditions.

### 1. Materials and Methods

Lyophilized chicken meat samples were pulverized using a mortar and stored in 250 mL beaker glasses.
Acetic acid (CAS 7647-01-0),
Sodium acetate (CAS 127-09-3),
Sodium phosphate monobasic (CAS 10028-24-7),
Sodium phosphate dibasic (CAS 13472-35-0), and
Sodium chloride (CAS 7647-14-5).

The solubility of lyophilized meat was tested using the following factors and concentrations based on previous analysis: 0.2 to 1 M of NaCl, 2 to 10% by weight of glucose, at pH ranges of 4 to 8 (0.05 M).

The matrix was generated using Design Expert 13 software trial version, and the design is a full factorial 23 with two replicates and 3 center points as laid out in Table 5.

TABLE 5
Design of experiments (Design Expert 13)

| | | Factor 1 A: | Factor 2 B: | Factor 1 C: |
|---|---|---|---|---|
| | | Salt | Glucose | PH |
| Run | Hours | (M) | (% by weight) | (0.05M) |
| 1 | 2 | 0.2 | 2 | 4 |
| 2 | 7 | 1 | 10 | 4 |
| 3 | 18 | 0.6 | 6 | 6 |
| 4 | 10 | 0.2 | 2 | 8 |
| 5 | 14 | 0.2 | 10 | 8 |
| 6 | 4 | 1 | 2 | 4 |
| 7 | 5 | 0.2 | 10 | 4 |
| 8 | 3 | 1 | 2 | 4 |
| 9 | 1 | 0.2 | 2 | 4 |
| 10 | 8 | 1 | 10 | 4 |
| 11 | 16 | 1 | 10 | 8 |
| 12 | 9 | 0.2 | 2 | 8 |
| 13 | 19 | 0.6 | 6 | 6 |
| 14 | 17 | 0.6 | 6 | 6 |
| 15 | 11 | 1 | 2 | 8 |
| 16 | 6 | 0.2 | 10 | 4 |
| 17 | 13 | 0.2 | 10 | 8 |
| 18 | 12 | 1 | 2 | 8 |
| 19 | 15 | 1 | 10 | 8 |

TABLE 6
pH Stock Solutions (2X)

| Buffer No. | | |
|---|---|---|
| 1 | pH 4 | Acetate buffer |
| | Stock solution (0.1M) | 40 mL |
| | Acetic acid | 18.52 |
| | Sodium acetate | 1.48 |
| 2 | pH 6 | Phosphate buffer |
| | Stock solution (0.1M) | 40 mL |
| | Sodium phosphate monobasic | 16.3 |
| | Sodium phosphate dibasic | 3.7 |
| 3 | pH 8 | Phosphate buffer |
| | Stock solution (0.1M) | 40 mL |
| | Sodium phosphate monobasic | 1.06 |
| | Sodium phosphate dibasic | 18.94 |

TABLE 7
NaCl Stock solution in different pH (2X)

| NaCl Molarity | pH (stock | NaCl | Volume |
|---|---|---|---|
| (M) | solution) | (g) | (mL) |
| 0.4 | 4 | 0.47 | 20 |
| 0.4 | 8 | 0.47 | 20 |
| 1.2 | 6 | 1.40 | 20 |
| 2 | 4 | 2.34 | 20 |
| 2 | 8 | 2.34 | 20 |

TABLE 8
Glucose Stock Solution (2X)

| Concentration | Glucose | Volume |
|---|---|---|
| (%) | (g) | (mL) |
| 4 | 0.8 | 20 |
| 12 | 2.4 | 20 |
| 20 | 4 | 20 |

### Chicken Powder Solubilization

For testing the protein solubility, as an example, a concentration of 100 mg/mL of meat powder was selected based on literature report. Approximately 500 mg of powder were homogenized in 5 mL of solutions according to Table 5 (see above). The solutions were briefly vortexed and homogenized in tube rotator for 1 hour. For homogenization, a tube rotator Stuart Tube Rotator SB3 at a speed of 14 rpm was used. After homogenization, the tubes were centrifuged and 500 μL supernatant was collected for Bradford assay. For centrifugation, a 5810R Eppendorf centrifuge was used at a relative centrifugal force (RCF) of 3214 for 10 min at 4° C.

### Bradford Assay

Quick Start™ Bradford Protein Assay was performed in a 96-well plate. The procedure was performed according to the instruction manual of the manufacturer of the assay kit (Bio Rad). The samples were properly diluted to in order to meet the requirement of protein limit detection concentration for Bradford assay. Protein concentration results were further analyzed. The results are depicted in Table 9.

### 2. Results

TABLE 9
Results of Bradford assay performed in duplicate. Herein,
the Run corresponds to that of Table 5 above and CM
refers to a control sample of MilliQ water

| | | | Mean Result | | | Dilution | Adj Result |
|---|---|---|---|---|---|---|---|
| Run | Value | Result | (mg/mL) | SD | CV | Factor | (mg/mL) |
| 1 | 0.387 | 0.444 | 0.462 | 0.027 | 5.7 | 50 | 23.121 |
| | 0.413 | 0.481 | | | | | |
| 2 | 0.418 | 0.489 | 0.52 | 0.044 | 8.5 | 50 | 25.986 |
| | 0.463 | 0.551 | | | | | |
| 3 | 0.464 | 0.552 | 0.49 | 0.087 | 17.8 | 50 | 24.524 |
| | 0.376 | 0.429 | | | | | |
| 4 | 0.384 | 0.44 | 0.45 | 0.014 | 3 | 50 | 22.497 |
| | 0.398 | 0.46 | | | | | |
| 5 | 0.424 | 0.497 | 0.535 | 0.054 | 10.1 | 50 | 26.74 |
| | 0.478 | 0.573 | | | | | |
| 6 | 0.429 | 0.504 | 0.537 | 0.046 | 8.6 | 50 | 26.846 |
| | 0.476 | 0.57 | | | | | |
| 7 | 0.384 | 0.44 | 0.444 | 0.006 | 1.4 | 50 | 22.215 |
| | 0.39 | 0.449 | | | | | |
| 8 | 0.418 | 0.488 | 0.475 | 0.018 | 3.8 | 50 | 23.741 |
| | 0.4 | 0.462 | | | | | |
| 9 | 0.311 | 0.338 | 0.381 | 0.061 | 16 | 50 | 19.03 |
| | 0.372 | 0.424 | | | | | |
| 10 | 0.505 | 0.611 | 0.634 | 0.033 | 5.3 | 50 | 31.72 |
| | 0.539 | 0.658 | | | | | |
| 11 | 0.44 | 0.519 | 0.553 | 0.048 | 8.7 | 50 | 27.65 |
| | 0.488 | 0.587 | | | | | |
| 12 | 0.442 | 0.522 | 0.564 | 0.06 | 10.7 | 50 | 28.22 |
| | 0.502 | 0.607 | | | | | |
| 13 | 0.437 | 0.514 | 0.556 | 0.059 | 10.7 | 50 | 27.805 |
| | 0.496 | 0.598 | | | | | |
| 14 | 0.464 | 0.552 | 0.56 | 0.01 | 1.8 | 50 | 27.988 |
| | 0.474 | 0.567 | | | | | |
| 15 | 0.477 | 0.571 | 0.583 | 0.017 | 3 | 50 | 29.151 |
| | 0.494 | 0.595 | | | | | |
| 16 | 0.402 | 0.465 | 0.492 | 0.037 | 7.6 | 50 | 24.587 |
| | 0.439 | 0.518 | | | | | |
| 17 | 0.413 | 0.481 | 0.527 | 0.066 | 12.5 | 50 | 26.36 |
| | 0.479 | 0.574 | | | | | |
| 18 | 0.515 | 0.625 | 0.616 | 0.013 | 2 | 50 | 30.825 |
| | 0.503 | 0.608 | | | | | |
| 19 | 0.5 | 0.604 | 0.624 | 0.029 | 4.7 | 50 | 31.223 |
| | 0.53 | 0.645 | | | | | |
| CM | 0.289 | 0.306 | 0.289 | 0.023 | 8.1 | 50 | 14.459 |
| | 0.265 | 0.273 | | | | | |

### 3. Conclusion

The results indicated at 95% of confidence interval. The NaCl concentration could influence solubility of the cell-containing material.

### Example 12 Biocompatibility and Absence of Toxicity

It was assured that the cell-based material in accordance with the present invention does not bear any toxic effects and is biocompatible and, thus, ingestible. For this purpose, a cellular assay was established to assess toxicity of a composition according to the present invention (here: “liquid meat”) during various production stages. Instead of standard 3T3 rodent cells, human HepG2 cells isolated from liver tissue were used, commonly applied to study drug metabolism and hepatotoxicity. Two different assays were evaluated to measure cytotoxicity and cell viability:

The Neutral Red Assay is an established method validated by the EURL ECVAM in the context of 3T3 cells. It is a cellular assay that is based on a eurhodin dye that stains the lysosomes of viable cells.

The CellTiter Glo assay is a viability assay commercialised by Promega. This cellular assay quantitates the amount of ATP that can be correlated to cell viability.

Both assays were setup using Doxorubicin, a strong chemotherapy agent intercalating with DNA and inhibiting topoisomerase III, an enzyme essential for DNA transcription.

As a next step, the toxicity of resubstituted lyophilized cell lysate was tested, where an early production step of a composition according to the present invention (here: “liquid meat”) was used. This sample was not further processed and thus contained a high amount of insoluble cell fraction.

### 1. Material and Methods

### Cell Culture

HepG2 cells were supplied by ATCC (HB-8065), cultured in DMEM supplemented with 10% FBS and grown in a incubator set to 37° C. and 5% CO₂. For both assays, cells were seeded at 5.000 cells/well in a 96-well plate using only the 32 central wells. Due to inherent clumping of HepG2 cells, amount of cells could not be properly measured and varied between assays. Various concentrations of Doxorubicin were added 18 hours after seeding to the cells at a volume of 0.2 μL of cells using IDOT. Cells were processed after 2 days of compound addition.

Time points for growth curves were measured every 3 hours recording 4 images/well using IncuCyte.

### Neutral Red Assay

The Neutral Red Assay Kit—Cell Viability/Cytotoxicity (ab234039, Abcam) was used. All solutions of the kit were aliquoted and stored at −20° C. as recommended. On the day of the experiment, aliquots of 20 mM Doxorubicin, washing solution, solubilization solution and Neutral Red Staining were thawed at room temperature (RT) and prepared according to the manufacturer's description. Cell medium was removed and cells washed with 200 μL of 1× washing solution. After addition of 150 μL of 1× Neutral Red Staining Solution, cells were stained for 2 hours in the incubator. Cells were washed with 250 μL of 1× washing solution, and then air dried for approx. 10 min until no liquid was visible on the plate. Addition of 150 μL 1× solubilization solution was followed by shaking the plate for 20 min at 500 rpm and RT. The 96-well plate was then read at a microplate reader at an OD of 540 nm.

### Cell Titer Glo Assay

The CellTiter-Glo 2.0 Cell Viability Assay (G9242, Promega) was used. The solution was thawed and stored at 4° C. as recommended. Cell medium was removed, then cells were washed 2× in 100 μL PBS. After the last wash, 50 μL of PBS followed by 50 μL of CellTiter-Glo 2.0 reagent were added. The 96-well plate was mixed for 2 min on an orbital shaker and incubated at 10 min at RT. Luminescence was recorded at a microplate reader, using a integration time of 1.000 ms. For both assays, data for dose-response experiments was fitted with the equation “Log(agonist) vs response−Variable slope (four parameters)” using Prism. For the dose-response curve of the Cell Titer Glo Assay, two data points were removed as outlier.

### 2. Results

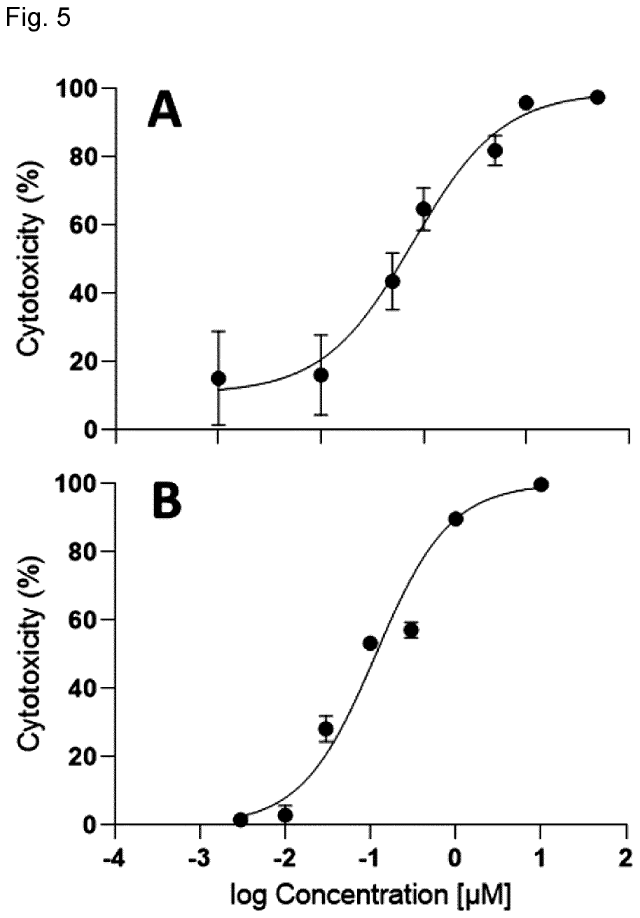

While the Neutral Red Assay showed a IC50 of 0.079 μM, CellTiter Glo 2.0 gave a IC50 of 0.116 μM. While this is higher than the expected literature value of 0.69 μM reported in the manual for the Neutral Red Assay with HepG2 cells, it is also not unexpected for a cellular assay to show this degree of variation. The results are depicted in FIG. 5.

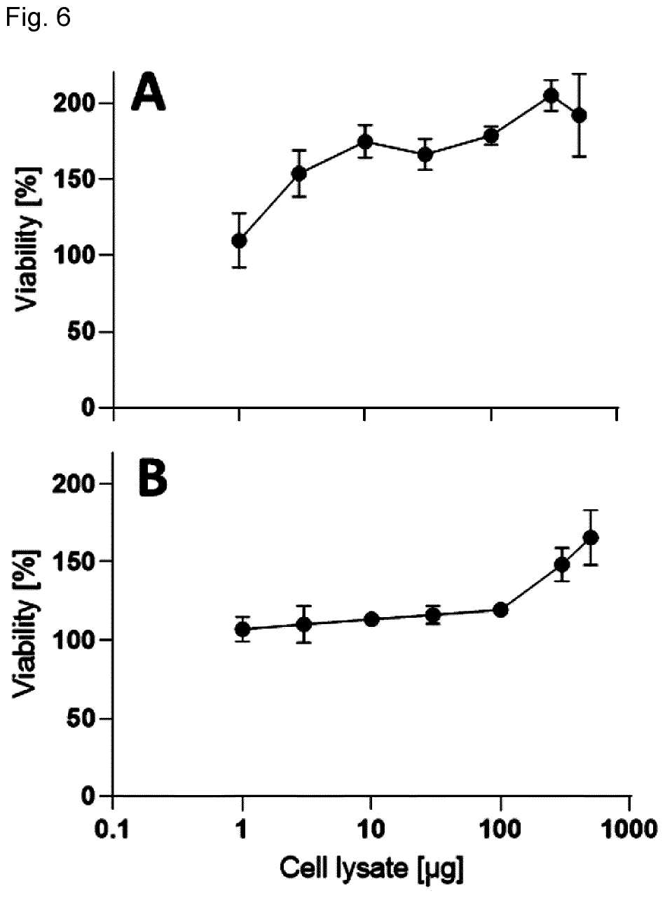

As a result, the Neutral Red Assay could not be used to assess toxicity because the particles interfered with the eurhodin dye. The CellTiter Glo was more successful and able to assess cell viability up to 100 μg of cell lysate depending on the number of cells (cf. FIG. 6). No toxic effect was observed. If the insoluble fraction should constitute an integral characteristic a composition according to the present invention (here: “liquid meat”) and is not to be removed by filtration or centrifugation, the CellTiter Glo assay can be used when the cell number in the assay is carefully adjusted and when the final cell lysate concentration is below 100 μg.

In summary, two assays with different methodologies to assess toxicity were set up, that can both be used without major adjustments to assess toxicity of small molecules. While the former is not able to detect toxicity in non-purified cell lysate, the latter shows compatibility up to 100 μg of protein cell lysate when carefully adjusted.

1. A method for preparing a comestible nutrient composition comprising:
(i) cultivating proliferating non-human animal cells of interest in vitro until a cell count of said cells has multiplied by at least 2-fold or more;
(ii) harvesting the cells obtained in (i) to provide cell-derived material; and
(iii) preserving the cell-derived material from the cells harvested in (ii) from spoiling; and
(iv) optionally mixing the cell-derived material of (iii) with one or more further consumable ingredients selected from the group consisting of one or more vitamins, one or more minerals, one or more aroma compounds, one more food colors, one or more types of fibers, ethanol, acetic acid, carbonic acid, and combinations of two or more thereof.

2. The method of claim 1, wherein the cultivating in (i) is performed in absence of animal-derived serum.

3. The method of claim 1, wherein (iii) comprises processing the cell-derived material into a powder optionally by at least partly removal of liquid content or by lyophilization.

4. The method of claim 1, wherein (iii) comprises pasteurization, wherein the cell-derived material from the cells harvested in (ii) optionally still contains cell culture medium and is a liquid or pasty composition.

5. The method of claim 1, wherein the comestible nutrient composition is:
(a) a drinkable composition optionally containing a mass ratio of dead material derived from the proliferating non-human animal cells of interest:viable cells of the proliferating non-human animal cells of interest of at least 2:1 or higher;
(b) a powder or granulate which is suspendable in aqueous solutions and water optionally containing a mass ratio of dead material derived from the proliferating non-human animal cells of interest:viable cells of the proliferating non-human animal cells of interest of at least 2:1 or higher; or
(c) a liquid or pasty composition that contains cell culture medium and living and/or dead cells wherein optionally the cell-derived material derived from (ii) contains cell culture medium in which the cells were cultivated in (i), wherein the liquid or pasty composition is optionally pasteurized.

6. The method of claim 1, wherein the cells of interest are:
myocytes or precursor cells thereof, in particular satellite cells;
adipocytes or precursor cells thereof;
hepatocytes or precursor cells thereof;
induced pluripotent stem cells;
non-human embryonic stem cells;
or a combination of two or more thereof.

7. The method of claim 1, wherein the cells of interest are primary cells.

8. The method of claim 1, wherein the cells of interest are cells of a permanent cell culture.

9. The method of claim 1, wherein the method further comprises adjusting content ratios and types of the cells cultivated in (i) to obtain a defined nutrient content of interest.

10. The method of claim 1, wherein (ii) comprises administering a solution containing a citrate salt.

11. The method of claim 10, wherein the solution containing the citrate salt is a buffer solution.

12. The method of claim 10, wherein the solution containing the citrate salt is administered to the cells for 2-20 minutes before flushing the cells.

13. The method of claim 1, wherein (ii) comprises removal of cell culture medium optionally by means of centrifugation, filtration, dialysis, or rinsing adherent cells and an optional washing with one or more buffers.

14. The method of claim 1, wherein (ii) comprises maintaining at least parts of cell culture medium and pasteurization.

15. The method of claim 1, wherein (iv) is performed and comprises adding:
at least 0.01% by weight, referred to the comestible nutrient composition as a whole, of at least one food color;
at least 0.2% by weight, referred to the comestible nutrient composition as a whole, of carbonic acid to obtain a sparkling drink;
at least 5% by volume, referred to the comestible nutrient composition as a whole, of ethanol;
at least 0.01% by weight, referred to the comestible nutrient composition as a whole, of at least one pharmaceutically active ingredient; or
a combination of two or more thereof.

16. The method of claim 1, wherein the comestible nutrient composition is a drinkable composition, (iii) comprises processing the cell-derived material into a powder, and the method further comprises suspending the powder in an aqueous liquid.

17. The method of claim 1, wherein the method further comprises:
(a) fermenting the comestible nutrient composition;
(b) smoking the comestible nutrient composition;
(c) pickling a comestible good with a composition comprising the comestible nutrient composition optionally in combination with one or more further component optionally selected from the group consisting of at least 1% by weight, referred to the composition, of sodium chloride, at least 1% by weight, referred to the composition, of one or more types of sugar, at least 1% by weight, referred to the composition, of acetic acid, at least 0.5% by weight, referred to the composition, of ethanol, or at least 5% by weight, referred to the composition, and one or more types of edible oil; and/or
(d) thickening the comestible nutrient composition to obtain a syrup, optionally under addition of a sweetener to obtain a sweet syrup.

18. A comestible nutrient composition obtainable from a method of claim 1, wherein the nutrient composition is optionally:
(a) a drinkable composition;
(b) a powder or granulate composition;
(c) a gel; or
(d) a frozen or partly frozen composition,
wherein the comestible nutrient composition may optionally form part of a filling of a capsule or may optionally form part of a drink, a dairy product, a non-dairy cream, a sauce, or a bakery good.

19. (canceled)

20. (canceled)

21. A method of treating a patient suffering from dysphagia, comprising administering to the patient a comestible nutrient composition, which is (a) drinkable composition; (b) a powder or granulate composition; (c) a gel; or (d) a frozen or partly frozen composition in a dysphagia treating effective amount, wherein the comestible nutrient composition is produced according to the method of claim 1.

22. The method of claim 21, wherein the administering of the comestible nutrient composition is by enteral nutrition, by swallowing a drinkable form of the comestible nutrient composition, or a combination thereof.

23. (canceled)
